SEQUENCE LISTING (5 sequences)

SEQ ID NO: 1 - DNA, 2544 bp, Homo sapiens
gccttaatagctagctggatgaatgtttaacttctaggccaggcactactctgtcccaacaataagccctgtacattgggaaaggtgccgagacatgaactttggtcttctctgcaatccatctggagcattcactgacaacatcgactttgaagttgcactgacctggccagccctgccacttaccaggttggctctgtatggctaagcgttttctcctaaaatcccttgaaaactgtgagaagaccataagaagatcatatctttaattctatttcacaagtcacacaatattccaatcaaatacagatggttgagaaaagtcatccatcttccctccccaccctcccacagcccctcaaccactgccctgaaacttatatgctgttatccgcagctccatctggagcatcacagctactgtcaaccctgacgctctttctgaaaaaacaccggatggacatcagaactatttctttaaggatgttactgagccacacaggaaaacttgccttatgattttgaatgcacggatctgatttgactaaacatgataactagagaatcacccaatctactcccattttcaactctaaatcatcagagtgtctcaaatccaaagcacacacagaccagcctggccaacacggtgaaactccacccctactaaaagtataaaaattatccaggtgtggtggcgggcgcctgtaatccaagctacttgggagtctggaggcaggagaatcccttgaacctgggagatggaggttgcagtgagcagagatcacaccaccgcactctagcctgggccacaaatcaacaacaacaacaacaacaaaaaacaaagcgcacacagagactgaggtcctctttggcattgagaagatggctatgcaagtcccaactagcaagtgcaaacttcccagcttcacttctgccagtgtcccttcaccccttctcaaccccactgggaggcaggagggtgcttgacaataacagccttggcatcactctgccagggtgtaataggaactgttacaattctgagattctgtgtaagcactggcctttctgcctagaatgccttctcctctcttttttaactgcatgctcctatttatctttcaaagcccggaaaaaataacactgcacacgggaaatgctcccttcctactgcagtcatttagatgactctatgccattccattcatttctctttcctaccacagaagtgctttgagattttggagtcagactgcttgaacttgaatcctggccctctcatcagagacttgacttattttaggcaagttatataaccaattttacctcagttccttacccataaaatgggtctaatgagagtacctaccacacagaattttgatgaaaactgaatgagatgaaggcctttaaggcagtggtccccaaccctggggacacagacaggtaccattttgtggcctgttaggaactgggccacacagcaggaggtgagcagtgggtgagtgagatcagcgttatttacagctgctccccattgctcaccttactgcctgagctccacctcctgtcagatcagcagtggcattaaattctcatagcagcacaaaccctgtcatgaactgcacatgcgagggatctaggttgtgcgctccttatgagaatctaatgcctaatgacctgtcaccgtctcccatcacccctagatgggagtgtctagttgcaggaaacaagctcagggcttccactgattctacattatggtgagttgtataattatttcattatataatacaatgtaataataatagaaacacagtgcacaacaaatgtaatgtgcttgaatcatccccaaaccatcccagtccacggtcttccacattttgtcttttcacaaaattgtcttccacaaaactggtccctggtgccaaaaaggcttgggaccactgctttaaagcctttgcatagtgcttagaattgagggggaaaaaaaaaacaaaaacaatgtagctagttgctacaatcactatattggtgagtttcaaaaggaaaagaattctgtcccatttatgcttgagccttgagttgctaaccaagcctgacacaaaattactgttgaagggatgtgtgagtcctaattgaaatgaggcctcttaagggaattgtggaccaaaccccaagcaggcagaaagccgtatcttaattattgcaagtatttcaggcaaggtgtggatggccatttgaattcaagcagactaggacctgggatgagaaagaaggtgtgtacgtgacttgatctttgaactttagctcaccatctggaagaaggctgagtattctctgcactcacatagtagctaatgcctactccccagccacccacaattctttctgtaggaaggctcgctagaatactttgtgatattggatattagttccatattctactgtgtatcttagttcaaccaaattgtaatcatctgatatttatttcttttaatataaatataagtatattaagtcttaaaaaaaaaaaaaaaa

SEQ ID NO: 2 - DNA, 24 bp, Homo sapiens
tcactgacaacatcgactttgaag

SEQ ID NO: 3 - DNA, 23 bp, Homo sapiens
ggagaaaacgcttagccatacag

SEQ ID NO: 4 - DNA, 23 bp, Homo sapiens
ctggccagccctgccacttacca

SEQ ID NO: 5 - DNA, 180 bp, Homo sapiens
cccggatccgccttaatagctagctggatgaatgtttaacttctaggccaggcactactctgtcccaacaataagccctgtacattgggaaaggtgccgagacatgaactttggtcttctctgcaatccatctggagcattcactgacaacatcgactttgaagttgcactgacgggtgt

(19) United States
(12) Patent Application Publication
(10) Pub. No.: US 2011/0097271 A1
(43) Pub. Date: Apr. 28, 2011

(54) Colon Cancer Associated Transcript 1 (CCAT1) As A Cancer Marker

(75) Inventors: Aviram Nissan, Jerusalem (IL); Stella Mitrani-Rosenbaum, Jerusalem (IL); Herbert Rudolf Freund, Jerusalem (IL); Tamar Peretz-Yablonsky, Jerusalem (IL); Marina Roistacher, Givaat Zeev (IL); Lloyd J. Old, New York, NY (US); Gerd Ritter, New York, NY (US); Ali O. Gure, Ankara (TR)

(73) Assignees: HADASIT MEDICAL RESEARCH SERVICES & DEVELOPMENT LI, Jerusalem (IL); LUDWIG INSTITUTE FOR CANCER RESEARCH AND CANCER IN, New York, NY (US)

(21) Appl. No.: 12/867,035

(22) PCT Filed: Feb. 11, 2009
(86) PCT No.: PCT/IL2009/000163

§ 371 Date: Dec. 28, 2010

Related U.S. Application Data
(60) Provisional application No. 61/064,005, filed on Feb. 11, 2008.

(51) Int. Cl. A61K 49/00; C12Q 1/68; C07H 21/04; C12N 15/63; C12N 5/10

(52) U.S. Cl. 424/9.1; 435/6; 536/24.31; 536/24.33; 536/23.1; 435/320.1; 435/325

(57) ABSTRACT

The present invention relates to the identification, isolation, and sequencing of a unique nucleic acid transcript termed Colon Cancer Associated Transcript 1 (CCAT-1) that is specifically expressed in cancer cells, in particular in colon, rectal, and lung cancer, as well as in precancerous lesions. The present invention thus provides methods for diagnosing cancer by detecting the expression of CCAT-1 as well as isolated polynucleotides, compositions and kits for use in the detection methods of the invention.

### FIELD OF THE INVENTION

This invention relates to a cancer marker CCAT-1 and to the use of CCAT-1 in the diagnosis and imaging of cancer.

### BACKGROUND OF THE INVENTION

Adenocarcinoma of the colon and rectum (CRC) is a common disease affecting annually over a million people worldwide (1). Current CRC screening is based mainly on fecal occult blood testing and diagnosis is based on colonoscopy and biopsy. Current screening programs have some effect on reduction of CRC-related mortality (2) however more accurate screening and diagnostic modalities are needed. Chemotherapy agents alone or in combination with targeted therapy improved median survival in metastatic patients and reduced disease-recurrence in patients who underwent complete resection of CRC and are at high-risk for recurrence (patients with lymph node metastasis or unfavorable histology, (3)). Despite the major advancements in CRC therapy, about 50% of patients diagnosed with CRC will die of disease within 5 years of diagnosis.

CRC-specific molecules, not expressed in normal tissues, are potential targets for therapy. There are few molecular markers expressed uniquely in CRC and not in normal tissues. Mass screening using cDNA arrays did not result in identification of CRC related molecular markers that can be used for diagnosis and as targets for therapy.

### SUMMARY OF THE INVENTION

The present invention is based on the identification, isolation and sequencing of a unique nucleic acid transcript that is specifically expressed in cancer cells, in particular in colon, rectal cancer. According to its unique expression profile the molecule was termed Colon Cancer Associated Transcript 1 (CCAT-1). The complete sequence of the molecule is disclosed herein below and is designated SEQ ID NO: 1

Subsequently, CCAT-1 was also found to be expressed in lung cancer. Accordingly, by a first of its aspects the present invention provides a method of diagnosing cancer or precancerous lesions, comprising measuring the level of expression of SEQ ID NO. 1 (CCAT-1) or a fragment thereof in a biological sample; wherein expression of SEQ ID NO. 1 (CCAT-1) or a fragment thereof in the biological sample, is indicative of cancer or a precancerous lesion.

In one embodiment, the method further comprises comparing said expression level measured in the biological sample with a standard, wherein a higher level of expression of SEQ ID NO. 1 (CCAT-1) or a fragment thereof in the biological sample, is indicative of cancer or a precancerous lesion.

In one embodiment, the method of the invention comprises:

(a) Isolating nucleic acids from a biological sample obtained from a subject;

(b) Hybridizing a probe capable of recognizing CCAT-1 with said nucleic acids, under conditions allowing the formation of hybridization complexes; and

(c) Comparing hybridization complex formation with a standard;

wherein a higher level of hybridization complexes in the biological sample is indicative of cancer or a precancerous lesion.

In another embodiment, the method of the invention comprises:

(a) isolating nucleic acids from a biological sample obtained from a subject;

(b) amplifying CCAT-1 or any fragment thereof in said isolated nucleic acids;

(c) visualizing the CCAT-1 amplified product; and

(d) comparing the amount of CCAT-1 amplified product with a standard;

wherein the presence of a higher level of a CCAT-1 amplified product is indicative of cancer or a precancerous lesion.

In a specific embodiment, said amplification is performed by polymerase chain reaction (PCR) using CCAT-1 specific probes. In a preferred embodiment, said PCR is a real-time quantitative PCR.

The term diagnosis of cancer or a precancerous lesion in accordance with the invention encompasses also staging of the cancer or a precancerous lesion, as well as in vivo imagining.

In accordance with one embodiment of the invention said standard is determined by measuring the level of expression of CCAT-1 in a subject not afflicted with cancer.

In another embodiment, said standard is determined by measuring the level of expression of CCAT-1 in a non-cancerous tissue of said same subject.

In accordance with certain embodiments of the invention the cancer is selected from the group consisting of: colon cancer, rectal cancer, lung cancer, and metastases of said cancers, including micro-metastases.

In accordance with another the embodiment the method of the invention diagnoses the precancerous lesion, adenomatous polyp.

In accordance with certain embodiments of the invention the biological sample is selected from the group consisting of tissue, blood, urine, stool, and bone marrow samples. Preferably, said biological sample is a tissue biopsy. The tissue biopsy may be obtained for example from the colon, rectum, liver, lung, and lymph nodes.

In another embodiment, the level of CCAT-1 expression is measured by in situ hybridization.

In another aspect, the present invention provides an isolated oligonucleotide comprising at least 8 contiguous nucleotides of SEQ ID NO: 1 (CCAT-1), or a complement thereof, preferably, for use as a probe or as a primer. In one specific embodiment, the oligonucleotide probe comprises SEQ ID NO: 5.

In accordance with certain embodiments, the isolated oligonucleotides of the invention are intended for use in detection of CCAT-1 expression in a biological sample.

In accordance with certain embodiments, the isolated oligonucleotides of the invention are intended for use in diagnosis of cancer.

In another aspect, the present invention provides a method for detecting the expression of CCAT-1 in a biological sample comprising:

(a) isolating nucleic acids from said biological sample;

(b) hybridizing the oligonucleotide probe of the invention to said nucleic acids under conditions allowing the formation of hybridization complexes; and

(c) comparing hybridization complex formation with a standard, wherein a higher level of hybridization complexes in the biological sample indicates expression of CCAT-1 in the sample.

In one embodiment, said method further comprises amplification of the transcribed nucleic acids of the biological sample prior to hybridization.

In another aspect, the present invention provides an isolated nucleic acid comprising CCAT-1 or a fragment thereof having at least 85% homology with SEQ ID NO: 1. In one embodiment, the isolated nucleic acid comprises the nucleic acid sequence of SEQ ID NO: 1 or a fragment thereof. In one embodiment the nucleic acid is mRNA. In another embodiment, the nucleic acid is cDNA.

In another aspect, the present invention provides compositions, including pharmaceutical compositions, comprising the isolated oligonucleotides, the isolated oligonucleotide probe, or the isolated nucleic acids of the invention as described above. In a specific embodiment, the compositions are attached to a substrate.

The invention further contemplates a vector comprising the isolated nucleic acids of the invention, as well as host cells comprising the vector.

In another aspect, the present invention provides a kit compartmentalized to receive at least one reagent for measuring CCAT-1 expression level in a biological sample obtained from a subject, said at least one reagent comprising at least one oligonucleotide probe or primer which hybridizes to at least a fragment of CCAT-1 transcript.

In yet another aspect, the present invention provides an array comprising a substrate having a plurality of segments, wherein at least one of said segments comprises a probe which hybridizes to CCAT-1 or a fragment thereof.

The present invention also contemplates imaging of cancer or precancerous lesions, including but not limited to adenomatous polyps, primary adenocarcinoma of the colon or rectum, lung cancer, lymph node metastasis and distant metastasis, by administering to a subject a probe capable of recognizing CCAT-1 wherein said probe is conjugated to an indicator molecule including but not limited to radio-isotope, fluorescent dye, visible dye or nano-particles and detecting the label by imaging devices.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 is a graph showing real-time PCR quantitative analysis of CCAT-1 transcript levels in a commercially available panel of cDNA from normal tissues. Left column represents CCAT-1 transcript levels in the colon cancer cell line SK-Co-10.

FIG. 2 is a graph showing real-time PCR analysis of CCAT-1 transcript levels in colon carcinomas (black bars) and adjacent normal tissue (white bars). Sample numbers are shown in the X-axes. C=control/SK-CO-10.

FIG. 3 is a graph showing a calibration curve for CCAT-1 in low concentrations of HT-29 cells. Amplification of CCAT-1 in increasing concentrations of HT-29 colon cancer cells diluted in peripheral blood mononuclear cells (PBMCs) obtained from subjects not afflicted with colon cancer. The relative quantity (RQ) of amplified CCAT-1 cDNA is shown in the y-axis and was calculated using a reference sample of PBMCs alone obtained from a subject not afflicted with colon cancer. The relative quantity of the CCAT-1 cDNA correlated with HT-29 tumor cell concentration. Lowest dose: five tumor cells in 1×10⁶ PBMCs up to 500 tumor cells (HT29) in 1×10⁶ PBMCs

FIG. 4 is a graph showing a calibration of intermediate concentrations of HT-29 cells. Amplification of CCAT-1 in increasing concentrations of HT-29 colon cancer cells diluted in PBMCs obtained from subjects not afflicted with colon cancer. The relative quantity (RQ) of amplified CCAT-1 cDNA was calculated using a reference sample of PBMCs alone obtained from a subject not afflicted with colon cancer. The relative quantity of the CCAT-1 cDNA (y-axis) correlated with HT-29 tumor cell concentration. Lowest dose: 500 tumor cells (HT29) in 1×10⁶ PBMCs up to 10,000 tumor cells (HT29) in 1×10⁶ PBMCs. The right column is the control, only PBMCs.

FIG. 5 is a graph showing calibration of high concentrations of HT-29 cells. Amplification of CCAT-1 in increasing concentrations of HT-29 colon cancer cells diluted in PBMCs obtained from subjects not afflicted with colon cancer. The relative quantity (RQ) of amplified CCAT-1 cDNA is shown in the y-axis and was calculated using a reference sample of PBMCs alone obtained from a subject not afflicted with colon cancer. The relative quantity of the CCAT-1 cDNA correlated with HT-29 tumor cell concentration. Lowest dose: 1,000 tumor cells in 1×10⁶ PBMCs up to 1,000,000 tumor cells (HT29) in 1×10⁶ PBMCs. The right column is a control sample, containing only PMBCs obtained from a subject not afflicted with colon cancer.

FIG. 6 is a graph showing expression of CCAT-1 in colonic adenomas (adenomatous polyps) and liver metastases derived from adenocarcinoma of the colon. 410TCo-tumor tissue, 316TCO-tumor tissue, NN=normal colonic mucosa obtained from patients with diseases other than colon cancer. P1 and P2=tissue obtained from adenomatous polyps of the colon. M=tissue obtained form liver metastases (metastatic colon cancer).

FIG. 7 is a graph showing the percentage of sentinel lymph nodes (SLNs) identified by the different detection methods including Hematoxillin and Eosin stain (H&E), immunohistochemistry (IHC) and polymerase chain reaction (PCR).

FIG. 8 is a graph showing an amplification plot of cytokeratin-20 (CK20) by real time quantitative PCR.

FIG. 9 is a graph showing an amplification plot of CCAT-1 by real time quantitative PCR (qPCR).

FIG. 10 is a graph showing results of qPCR for CCAT-1 in stool samples. The results are shown as the relationship between the expression of CCAT-1 and the housekeeping gene (GAPDH). Each column represents the relative quantity (RQ) of CCAT-1 cDNA compared to the quantity of GAPDH cDNA amplified from the same sample. NTC (the left column) shows the internal negative control—peripheral blood lymphocytes obtained from a subject not afflicted with colon cancer. No CCAT-1 cDNA is present. Samples t391 and t374 are positive controls—primary adenocarcinoma of colon tissues. These samples show high expression of CCAT-1. Stool samples from healthy volunteers: (C46-C8). No expression of CCAT-1 in any of the samples (n=9). Stool samples of patients with adenocarcinoma of the colon or rectum: (P25-P1). CCAT-1 Expression was found in 4/12 samples.

FIG. 11 is a graph showing expression of CCAT-1, measured by real-time PCR quantitative analysis, in peripheral blood of nine negative controls (healthy volunteers) and in two samples of Adencarcinoma of the colon (left columns).

FIG. 12 is a graph showing expression of CCAT-1 in peripheral blood samples of colon cancer patients. The relative quantity (RQ; shown in the y-axis) of amplified CCAT-1 cDNA compared to control sample of PBMCs obtained from a subject not afflicted with colon cancer (NC).

FIG. 13 a graph showing relative CCAT-1 expression in 18 colorectal cancer cell lines compared to HT-29. Expression level was determined by real-time (quantitative) PCR. The relative CCAT-1 expression in any given sample was compared to CCAT-1 expression in HT-29 colon cancer cell line (=1).

FIG. 14 is a graph showing relative CCAT-1 expression in 16 lung cancer cell lines compared to HT-29. Expression level was determined by real-time (quantitative) PCR. The relative CCAT-1 expression in any given sample was compared to CCAT-1 expression in HT-29 colon cancer cell line (=1).

FIG. 15A shows a 165 bp nucleic acid sequence utilized as an in-situ probe (SEQ ID NO: X); FIG. 15B is a photograph showing Northern blot analysis of a pBluescript vector. Lane 1: ladder. Lane 2: pBluescript vector and the insert.

FIG. 16 is an exemplification of In situ hybridization based screening. Comparative staining of CCAT-1 in adenocarcinoma of the colon (a) and in adjacent normal mucosa (b). Stronger CCAT-1 staining is seen in the tumor tissue (a) as compared to a lower intensity of the CCAT-1 staining in adjacent normal mucosa.

FIG. 17 is a graph showing expression of CCAT-1 in peripheral blood samples of healthy volunteers.

### DETAILED DESCRIPTION OF EMBODIMENTS

### Definitions

As used herein, the term “CCAT-1” refers to Colon Cancer Associated Transcript 1.

As used herein, the term “fragment of a CCAT-1” or “fragment of a CCAT-1 transcript” refers to fragments of CCAT-1 gene transcript which may be useful as an oligonucleotide or nucleic acid probe, a primer, or an antisense oligonucleotide.

As used herein, the terms “polynucleotide” and “oligonucleotide” are used interchangeably, and include polymeric forms of nucleotides of any length, either deoxyribonucleotides or ribonucleotides, or analogs thereof. The following are non-limiting examples of polynucleotides: a gene or gene fragment, exons, introns, messenger RNA (mRNA), transfer RNA (tRNA), ribosomal RNA (rRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The term also includes both double- and single-stranded molecules.

A “complementary transcript” or a “probe” for CCAT-1 refers to a nucleic acid molecule or a sequence complementary therewith, used to detect the presence of at least a portion of the cDNA or an mRNA of CCAT-1. The detection is carried out by identification of hybridization complexes between the probe and the assayed sequence. The probe can be attached to a solid support or to a detectable label. The probe will generally be single stranded. The probe(s) typically comprise 10 to 200 nucleotides. The particular properties of a probe will depend upon the particular use and are within the competence of one of ordinary skill in the art to determine. Generally, the probe will hybridize to at least a portion of the cDNA or an mRNA of CCAT-1 under conditions of high stringency

As used herein, a “primer” refers to a short polynucleotide that binds to a target or “template” target present in a sample of interest by hybridizing with the target, and thereafter promoting polymerization of a polynucleotide complementary to the target.

A “polymerase chain reaction” (PCR) is a reaction in which replicate copies are made of a target polynucleotide using a set of primers, typically a pair of primers, consisting of a forward and a backwards primer, and a DNA polymerase as a catalyst of polymerization.

It should be understood that a primer can also be used as a probe in hybridization reactions, such as Southern or Northern blot analyses as described in, for example, Sambrook J. et al: A Laboratory Manual. 2nd, ed., Cold Spring Harbor Laboratory, Cold Spring Harbor Laboratory Press, Cold Spring Harbor, N.Y., 1989.

As used herein the term “cDNA” refers to complementary DNA. “cDNA” refers to an isolated polynucleotide, nucleic acid molecule, or any fragment or complement thereof. It may have originated by recombinant techniques or synthetically, be double-stranded or single-stranded, represent coding and/or non-coding 5′ and 3′ sequences. As used herein, “colorectal cancer” or “CRC” refers to a medical condition characterized by cancer of cells of the intestinal tract including cecum, ascending colon, transverse colon, descending colon, sigmoid colon, and rectum.

As used herein, “precancerous lesion” or “adenomatous polyp” refers to a medical condition characterized by malignant transformation of the colonic mucosa without histological evidence of invasion into the basement membrane.

As used herein, “lung cancer” refers to a medical condition characterized by cancer of cells of the lung.

A “vector” includes a self-replicating nucleic acid molecule that transfers an inserted polynucleotide into a host cell. The term is intended to include vectors that function primarily for insertion of a nucleic acid molecule into a cell, replication vectors that function primarily for the replication of nucleic acid and expression vectors that function for, transcription and/or translation of the DNA or RNA. Also intended are vectors that provide more than one of the above functions.

The term “host cell” encompasses any individual cell or cell culture which serves as a recipient for vectors or for the incorporation of exogenous nucleic acid molecules such as polynucleotides. It particular, host cell encompasses a cell capable of serving as a recipient for vectors comprising at least a portion of CCAT-1. It also encompasses progeny of a cell which sometimes may not necessarily be identical in its genotype or phenotype to the original parent cell due to natural, accidental, or deliberate mutation. Prokaryotic or eukaryotic cells are suitable to serve as a host cell in the present invention. Host cells include, but are not limited to bacterial cells, yeast cells, insect cells, animal cells, including mammalian cells, e.g., murine, rat, simian or human cells.

As used herein “differential expression” refers to an increased, higher (upregulated or present), or a decreased (downregulated or absent) gene expression as detected by the absence, presence, or changes in the amount of transcribed oligonucleotides (e.g. mRNA) in a biological sample. “Higher expression level” encompasses an expression level that is at least 2 times, 2.5 times, 3 times, 5 times 10 times, or more, higher than the expression level detected in a control sample or a standard. This term also refers to expression of nucleotide sequences in a cell or tissue while no expression is detected in a control cell.

As used herein, “hybridization” refers to a reaction in which at least one polynucleotide reacts to form a complex that is stabilized via hydrogen bonding between the bases of the nucleotide residues. The hydrogen bonding may occur by Watson-Crick base pairing, in any other sequence-specific manner. A hybridization reaction may constitute a step in a more extensive process, such as the initiation of a PCR reaction.

Hybridization reactions can be performed under conditions of different stringency. Under stringent conditions, nucleic acid molecules at least 60%, 65%, 70%, 75% identical to each other remain hybridized to each other. A non-limiting example of highly stringent hybridization conditions is hybridization in 6× sodium chloride/sodium citrate (SSC) at approximately 45° C., followed by one or more washes in 0.2×SSC and 0.1% SDS at 50° C., at 55° C., or at about 60° C. or more.

When hybridization occurs in an antiparallel configuration between two single-stranded polynucleotides, those polynucleotides are described as complementary.

The molecules capable of hybridizing with the nucleic acid molecules of the invention, SEQ NO: 1 (CCAT) also comprise polynucleotide fragments capable of hybridizing thereto. Herein, fragments are understood to mean parts of the nucleic acid molecules which are long enough to hybridize to CCAT-1 transcript. Therefore, the term fragment means that the sequences of these molecules differ from the sequences of CCAT-1 transcript in one or more positions and still show a high degree of homology to these sequences or a part thereof. In this context, homology means a sequence identity of at least 40%, in particular an identity of at least 60%, at least 80%, at least 85%, at least 90% or more than 95%.

Preferably, the degree of homology is determined by comparing the respective sequence with the nucleotide sequence of SEQ ID NO: 1. In such a case where the sequences which are compared do not have the same length, the degree of homology preferably refers to the percentage of nucleotide residues in the shorter sequence which are identical to nucleotide residues in the longer sequence. The degree of homology or identity can be assessed, for example, by known computer alignment based programs such as the ClustalW which is distributed by the European Bioinformatics Institute (EBI) and the European Molecular Biology Laboratory (EMBL). ClustalW can be downloaded from various sources such as for example: www.ebi.ac.uk/clustalw. When using ClustalW package version 2.0 to determine whether a particular sequence is, for example, 85% identical to the SEQ NO: 1 according to the present invention, the comparison can by performed by the default settings provided therein.

A “biological sample” is used herein in a broad sense and may comprise a bodily fluid; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a biopsy, a tissue including tumor tissue, colorectal sample and non-colorectal samples; a tissue print; a fingerprint, buccal cells, skin, or hair; and the like.

A “Substrate” refers to any rigid or semi-rigid support to which nucleic acids are bound and includes membranes, filters, chips, slides, wafers, fibers, magnetic or nonmagnetic beads, gels, capillaries or other tubing, plates, polymers, nano-particles, and microparticles with a variety of surface forms including wells, trenches, pins, channels and pores.

The present invention is based on the identification of Colon Cancer Associated Transcript-1 (CCAT-1), a novel non-coding RNA which is 2528 base pairs (bp) long, and which is located on chromosome 8 q 24.21.

CCAT-1 was found by the inventors to be present in various cancerous tissues and cell lines including colon, rectal, lung cancer, and in precancerous lesions (adenomatous polyps) in the colon but was detected at very low levels, if at all, in normal tissues as disclosed herein. CCAT-1 can therefore serve as a marker for cancer cells and is useful in particular in the identification of colorectal, lung cancer, and precancerous lesions (adenomatous polyps) in a biological sample.

Thus, the present invention provides the use of CCAT-1 or any part of its sequence as a specific molecular diagnostic marker for the diagnosis, staging (pathological evaluation), imaging and post-operative surveillance of patients with colorectal cancer (CRC), precancerous lesions (adenomatous polyps) and lung cancer.

These activities may be performed in vitro, or in vivo by delivery of compositions for imaging a tumor in the patient.

Detection can be achieved using polymerase chain reaction (PCR), in-situ hybridization techniques, or any other detection technique as known in the art.

According to some embodiments of the invention, compositions and methods are provided for screening, diagnosing and analyzing patients and/or patient samples to detect evidence of CCAT-1 expression. The expression of CCAT-1 is suggestive of primary or metastasized colorectal cancer or primary or metastatic lung cancer. In an additional aspect of the invention, compositions and methods are provided which are useful to visualize primary or metastasized colorectal cancer or primary or metastatic lung cancer cells.

Screening and diagnostic compositions and methods can be used in the monitoring of individuals who are in high risk groups for colorectal or lung cancer such as those who have been diagnosed in the past with localized disease, metastasized disease or those who are genetically linked to the disease, or those who have family members of first and second degree diagnosed in the past with cancer. Individuals with a history of inflammatory conditions of the colon such as ulcerative colitis or Crohn's colitis and individuals with a history of tobacco smoking will also be considered as individuals who are in high risk groups for colorectal or lung cancer. In vitro screening and diagnostic compositions, and methods can be used in the monitoring of individuals who are undergoing or have been treated for colorectal or lung cancer to determine if the cancer has been eliminated. Screening and diagnostic compositions and methods can be used in the monitoring of individuals who have been identified as genetically predisposed such as by genetic screening and/or family histories.

Accordingly, individuals who are at risk for developing colorectal and lung cancer may be identified and samples may be isolated from such individuals. The invention is useful for diagnosing individuals who show at least one symptom or characteristic of cancer, e.g. presence of a polyp in the relevant tissue. The invention is particularly useful for monitoring individuals who have been identified as having family medical histories which include relatives who have suffered from colorectal or lung cancer. Likewise, the invention is particularly useful to monitor individuals who have been treated and had tumors removed or are otherwise experiencing remission.

According to the invention, compounds are provided which bind to CCAT-1 gene transcript.

The detection of colon, rectal or lung cancer can be performed using any biological sample obtained from a cancer patient or an individual suspected of cancer including, but not limited to, tissue, blood, bone marrow, stool, urine, lymph nodes, or any body fluid. In a specific embodiment, the biological sample is a biopsy (e.g. needle biopsy or tissue removed during colonoscopy) taken from the tissue suspected of being cancerous. In a particular embodiment, the sample is a stool sample.

Tissue samples can be obtained by surgical techniques. The person skilled in the art would appreciate the plurality of test samples that may be obtain for analysis and examination according to the present invention. Additionally, the person skilled in the art would understand that present invention can use additional procedures for the purpose of obtaining tissue samples.

In a one embodiment, blood is used as the biological sample. If that is the case, the cells comprised therein can be isolated from the blood sample by centrifugation, for example.

In a preferred embodiment the biological sample is obtained from a human.

Detecting the presence of the CCAT-1 gene transcript in any of the biological samples suggests that the biological sample contains tumor cells.

Tissue samples are optionally homogenized by standard techniques e.g. sonication, mechanical disruption or chemical lysis.

Tissue section preparation for surgical pathology can be frozen and prepared using standard techniques. In situ hybridization assays on tissue sections are performed in fixed cells.

The presence of CCAT-1 gene transcript can be determined using various techniques known in the art, for example, Polymerase Chain Reaction (PCR) amplification of the gene transcript or a fragment thereof using specific primers and detection of the amplified product, and hybridization with CCAT-1 specific probes.

The presence of the CCAT-1 gene transcript can also be determined in tissue sections using techniques such as in situ hybridization.

To that end, the present invention provides oligonucleotide probes and oligonucleotide primers which are employed for identifying the CCAT-1 in the procedures of the present invention.

In another aspect, the present invention provides for kits which comprise such components as oligonucleotide probes and oligonucleotide primers. It should be appreciated that the examples provided herein are not meant to limit the scope of the invention.

As noted above, detecting the CCAT-1 gene transcript in a biological sample can be performed using polymerase chain reaction (PCR) technology. PCR is known in the art and routinely practiced both in diagnostics and in research. It is disclosed, for example in U.S. Pat. Nos. 4,683,202, and 5,075,216. In brief, PCR provides for the amplification of DNA/RNA sequences by providing primers that hybridize to the target nucleotide sequence to be amplified. A set of primers generally contains two primers (+forward and − backward). The reaction also employs nucleotides and a polymerase enzyme. The polymerase fills in complementary nucleotides according to the sequences adjacent to the hybridized primers. Amplification cycles result in exponential amplification of the desired product.

PCR primers are designed according to routine protocols on the basis of sequence information. The nucleotide sequence of the CCAT-1 transcript is set forth in SEQ ID NO: 1.

For PCR, the RNA is typically extracted from cells in the biological sample and analyzed or alternatively transformed to cDNA. Such transformation is also standard practice.

When a CCAT-1 transcript is present in the biological sample, PCR will result with exponential copies of the original mRNA comprising the CCAT-1 transcript.

If CCAT-1 is absent, PCR will not result in a detectable amplification product. Primers suitable for PCR are generally 8-25 nucleotides long, 15-30 nucleotides long, or 18-50 nucleotides long. Typical primers comprise 15-25 nucleotides. These primers are identical or complement the sequence of CCAT-1 transcript or its counterpart cDNA. This is the reason why these primers hybridize to the CCAT-1 transcript or its fragments.

The mRNA or its cDNA counterpart which were previously obtained from the biological sample are mixed with the primers, the nucleotides and polymerase enzyme following known protocols of PCR. The mixture undergoes a series of temperature cycles. When the CCAT-1 transcript or its corresponding cDNA is present in the mixture, the primers will hybridize, and the CCAT-1 transcript will be exponentially amplified. When the CCAT-1 transcript is absent, no detectable amplification would be observed. The amplified product can be detected by numerous procedures well known in the art, for example, by gel electrophoresis.

PCR is favorable when small amounts of transcribed polynucleotides are recovered from the biological sample.

The present invention further provides polynucleotide primers suitable for PCR reactions aimed at amplifying the CCAT-1 transcript.

According to the invention, diagnostic kits useful for detecting the presence of the CCAT-1 transcript in a biological sample can be assembled. Such diagnostic kits comprise reagents suitable for the detecting CCAT-1. By way of non-limiting example, such kits comprise at least one oligonucleotide probe and/or at least one oligonucleotide primer. Typically, kits of the present invention further comprise a container for the reagents used. Additionally, these kits can comprise nucleotide size markers for use in gel analysis in order to determine the size of the detected nucleic acids. Kits of the present invention can additionally include instructions and protocols for performing the assays. Positive and negative controls may also be provided as a part of the kit assemblies.

In another embodiment, the determination whether a sample contains cells expressing CCAT-1 is by Northern blot analysis of mRNA extracted from a biological sample. Northern blot analysis is a method known to the person skilled in the art. See e.g. Sambrook et al., (1989) Molecular Cloning: A Laboratory Manual, Cold Spring Harbor Laboratory Press, Cold Spring Harbor, N.Y.

Additionally, mRNA extraction, mRNA separation by electrophoresis, blot analysis, probe and primer preparations and hybridization techniques are known and material for carrying out these techniques is commercially available.

Messenger RNA could be extracted, for example, by using poly dT columns and the material is separated by electrophoresis and, for example, transferred to nitrocellulose paper.

Labeled probes are commonly used to visualize the presence of mRNA fixed to the paper. These probes have a nucleotide sequence that is complementary to the CCAT-1 transcript or a fragment thereof.

To that end, the sequence information in SEQ ID NO: 1 can be used to prepare the probes or to isolate and clone the CCAT-1 transcript. Such probes are at least 8, 15, 30, 40, or 100 nucleotide stretches. The probe may be DNA or RNA, it is preferably single stranded and typically should have at least 65% sequence homology to the corresponding fragment of SEQ ID NO: 1.

The probes may be produced using oligolabeling, or PCR amplification in the presence of a reporter molecule. A vector containing the cDNA of CCAT-1 or a fragment thereof may be used to produce a messenger RNA probe, for example, by addition of an RNA polymerase and a labeled nucleotide. The person skilled in the art can carry out these processes with commercially available kits and materials.

The stringency of hybridization is determined by numerous factors such as GC content within the probe, temperature and salt concentration. In particular, stringency can be increased by reducing the concentration of salt or raising the hybridization temperature. At high stringency, hybridization complexes will remain stable only where the nucleic acids are highly complementary.

Conditions for hybridization are well known to the person skilled in the art e.g. Sambrook et al, noted above.

The kits of the present invention can therefore contain useful reagents to practice Northern blot techniques for detecting the presence of the CCAT-1 transcript in a biological sample. The kits optionally comprise oligonucleotides which can be used as probes for hybridizing to the transcribed CCAT-1 or a fragment thereof. The probes can be radiolabeled. Positive and negative controls can be also provided optionally together with appropriate size marker.

Another technique for detecting the presence of the CCAT-1 transcript is by way of oligonucleotide hybridization. Hybridization of polynucleotides is known to the person skilled in the art. This method also employs detectable probes which contain a specific nucleotide sequence that hybridizes to nucleotide sequence of the CCAT-1 transcript.

RNA from a biological sample is fixed, typically to filter paper or the like. The probes are added and maintained under conditions which allow hybridization only if the probes appropriately complement the fixed material.

These conditions should be sufficiently stringent to wash off partially hybridizing probes to the fixed material. Detection of the probe on the filter indicates the presence of CCAT-1 transcript.

Probes for hybridization assays comprise at least 8, 12, 15, 20, 30, 50 or 100 nucleotides complementary to the sequence to the CCAT-1 gene transcript.

The sequence information disclosed in SEQ ID NO: 1 can be used by the person skilled in the art to prepare probes of the invention. The condition for the hybridization process can be optimized to minimize background signal caused by non-fully complementary polynucleotides in the sample.

The present invention therefore further includes labeled oligonucleotides which are useful as probes for oligonucleotide hybridization. Labeled probes encompass radiolabeled nucleotides or otherwise detectable probes by readily available systems. Labeled probes typically incorporate a label measurable by spectroscopic, photochemical, biochemical, immunochemical, or chemical means. By way of non-limiting example, such labels can comprise radioactive substances (³²P, ³⁵S, ³H, ¹²⁵I), fluorescent dyes (digoxigenin, fluorescein, 5-bromodesoxyuridin, acetylaminofluorene), biotin, nanoparticles, and the like. Such oligonucleotides are typically labeled at their 3′ and 5′ ends.

In particular, a transcript complementary to the CCAT-1 (for example SEQ ID No: 4) or any part of its sequence may be conjugated to a fluorescent probe including, but not limited to, fluorescent tags in the visible range (CY3.0 or CY 5.0 for example) or probes in the near infrared range (Cy 5.5 for example) for in-vitro or in-vivo detection or imaging of colon or rectal cancer, and lung cancer. Additionally, a transcript complementary to the CCAT-1 or any part of its sequence may be conjugated- to or incorporated—in nano particles, micro-particles, or liposomes for detection or imaging of cancer.

A transcript complementary to the CCAT-1 or any part of its sequence may be conjugated to radioactive isotopes for detection or imaging of cancer. A transcript complementary to the CCAT-1 or any part of its sequence may be conjugated to synthetic polymers for detection or imaging of cancer.

A transcript complementary to the CCAT-1 or any part of its sequence may be conjugated to other biologic agents including but not limited to antibodies, toxins, or other peptides for detection or imaging of cancer.

Kits can be assembled which are useful to carry out the hybridization methods of the invention. These kits further provide labeled oligonucleotides which hybridize to the CCAT-1 transcript. In one embodiment, the labeled probes are radiolabeled. Positive and negative controls can further be included in said kits as well as size markers such as a polynucleotide ladder. These kits can further comprise instructions for performing the assay.

The hybridization technique of the present invention further encompasses in situ hybridization to detect cells that express CCAT-1 in a biological sample such as tissue sections. Therefore, the present invention further relates to probes which are useful for carrying out in situ hybridization. These probes are designed to hybridize to the complementary nucleic acid sequences present in a biological sample. Fluorescent microscope can be utilized for visualization of probes labeled with fluorescent markers.

To that end, the present invention also provides kits for performing in situ hybridization. In situ hybridization can be used to detect the mRNA sequence of CCAT-1 in a tissue section. A fluorescent marker can be used to detect the sequence corresponding to the CCAT-1 transcript or a complementary sequence thereof.

The present invention further relates to recombinant vectors, including expression vectors that comprise the CCAT-1 gene transcript, a fragment thereof, or a complement thereof.

The present invention further relates to host cells which comprise such vectors and to methods of expressing CCAT-1 using such recombinant cells. Examples of host cells include yeast cells such as S. cerevisiae, insect cells such as S. frugiperda, bacteria such as E. coli, and mammalian cells such as Chinese Hamster Ovary (CHO) cells.

The present invention also provides for an isolated oligonucleotide comprising the sequence of CCAT-1 gene transcript (SEQ ID NO: 1) or a fragment thereof. In particular, the present invention is directed to an isolated oligonucleotide having at least 85% homology with SEQ ID NO: 1. The present invention relates to the isolated CCAT-1 including a fragment thereof.

The recombinant expression vectors of the invention are useful for transforming hosts to prepare recombinant expression systems for preparing the isolated oligonucleotides of the invention. The expression vector can contain transcriptional control elements (e.g. promoters and enhancers). These elements can be selected from various sources which have been selected for their efficiency in a particular host. The vector, cDNA, and regulatory elements are combined using recombinant DNA techniques or synthetic techniques.

The person skilled in the art can introduce such molecules comprising the CCAT-1 polynucleotides of the present invention by use of commercially available expression vector for use in well known expression systems such as those described herein.

In addition to producing these polynucleotide molecules by recombinant techniques, automated nucleic acid synthesizers may also be employed to produce CCAT-1 or a fragment thereof. Such techniques are well known to those having ordinary skill in the art and.

The CCAT-1 transcript, cDNAs corresponding to CCAT-1, fragments thereof, oligonucleotides, complementary RNA and DNA molecules to any of the above, and also PNAs can be used to detect or measure differential CCAT-1 expression, increased expression levels. These measurements can monitor mRNA levels during therapeutic intervention. It can also be utilized in diagnostic procedures of the present invention, or indeed prognostic determinations. These measurements can also be utilized in staging cancer tissues such as CRC or lung cancer.

Cancers associated with differential expression include specifically colon or rectal cancer as well as lung cancer. The diagnostic assay may use hybridization or amplification technology as described above. They can be utilized in comparing gene expression in a biological sample obtained from a patient to a standard sample in order to detect differential gene expression or increased expression level of CCAT-1. Quantitative methods for this comparison are well known to the person skilled in the art. In particular, quantitative PCR (qPCR) or real time quantitative PCR (RT-qPCR) analysis can be used to quantify CCAT-1 expression levels in a biological sample. In brief, real time quantitative PCR provides for simultaneous monitoring over the amplification process. The amplification product is continuously detected as it accumulates as the procedure continues. Quantitative PCR and RT-qPCR is a procedure known to the person skilled in the art.

By way of a non-limiting example, a labeled probe may be mixed with nucleic acids prepared from a biological sample obtained from a patient. The mixture is maintained under conditions for the formation of hybridization complexes. After a particular incubation, the sample is washed and the amount of signal for the hybridization complexes is quantified. The signal can be also compared with a standard value. A higher signal of CCAT-1 in a biological sample obtained from an individual in comparison to a normal standard indicates cancer (e.g. CRC or lung cancer).

In order to provide standards for establishing differential expression or increased expression of CCAT-1, normal and disease expression levels are assessed. A nucleic acid sample taken from normal subjects is reacted with an oligonucleotide probe complementary to CCAT-1 under conditions that allow hybridization. Then, the amount of hybridization complexes is measured. Standard values obtained in this manner may be compared with values obtained from biological samples of the tested individuals.

The standard level may be determined by measurement of CCAT-1 expression in individuals not afflicted with cancer (termed “normal subjects”). Alternatively, the standard level can be determined according to the expression level of a reference gene in a biological sample, optionally of the tested individual. The reference gene can be a house-keeping gene such as exemplified herein (e.g. GAPDH). Differential CCAT-1 expression in a biological sample can therefore be identified by an increase in the ratio of CCAT-1 expression compared with the level of expression of the reference gene, or increase of an otherwise normalized CCAT-1 expression measurement.

The standard may also be determined by comparison of CCAT-1 levels in the tissue to a surrounding tissue considered to be cancer free (also termed “non cancerous tissue” or “normal tissue”).

In some embodiments, the CCAT-1 primers and/or probes of the invention may be formulated in a variety of forms including a solution, suspension, emulsion, or lyophilized powder. The formulation is sterilized by commonly used techniques.

In another aspect, the present invention provides an array of oligonucleotides comprising a substrate having a plurality of segments, wherein at least one of said segments comprises a probe which hybridizes to CCAT-1 or a fragment thereof. In a specific embodiment, the array comprises a probe of the present invention, e.g. SEQ ID NO: 5.

### EXAMPLES

The examples provided herein below illustrate the invention and are not intended to limit the scope of the subject invention.

### Example 1

### Discovery of CCAT-1

Patients, Cell Lines, Tissues and RNA

The colon cancer cell lines HT-29 and SK-CO-10 were obtained from ATCC (Manassas, Va.). HT29 was used to prepare tester RNA for Representational difference analysis (RDA) experiments. Colon carcinoma and adjacent normal tissue specimens were obtained either from the tissue bank maintained by the Ludwig Institute (#4 to 29) or at the Hadassah-Hebrew University Medical Center (#96-218). All samples were from patients undergoing surgical tumor resection and who signed a written informed consent approved by the Institutional Review Board (IRB). Total RNA was either obtained by the guanidium isothiocyanate/CsCl gradient purification method or the Trizol reagent (Sigma-Aldrich, St. Louis, Mo.). A panel of normal tissue RNA was obtained from Clontech (Mountain view, Calif.).

RDA and cDNA Cloning

Representational difference analysis (RDA) was done as previously described (4). HT29 constituted the tester RNA. For the driver, RNA was pooled from three normal colon tissues. Three rounds of amplification utilizing Tsp509I or DpnII as the restriction endonuclease were performed. Fragments generated after the second and third amplification cycles were isolated and sequenced. The partial length CCAT-1 transcript identified by RDA was used to screen a HT29 cDNA library in the λ-ZAPII vector system (Stratagene, La Jolla, Calif.) by which the full-length cDNA was obtained.

The CCAT-1 Gene Transcript and Expression

The full length CCAT-1 cDNA cloned from a HT29 cDNA library is 2528 bp long. The transcript corresponds to two exons spanning nts.1-194 and 195-2528, as revealed by a BLAST analysis against two chromosome-8 genomic clones (AC027531 and AC020688), with a long intron of about 9 kb. The cDNA is identical to AK125310, identified in teratocarcinoma (5). Compared to AK125310, CCAT-1 lacks 86 nucleotides at its 5′-end and has an additional 313 at the 3′.

The full sequence of CCAT-1 (SEQ ID NO. 1) is:

GCCTTAATAGCTAGCTGGATGAATGTTTAACTTCTAGGCCAGGCACTACT
CTGTCCCAACAATAAGCCCTGTACATTGGGAAAGGTGCCGAGACATGAAC
TTTGGTCTTCTCTGCAATCCATCTGGAGCATTCACTGACAACATCGAGCT
TTGAATTGCACTGACCTGGCCAGCCCTGCCACTTACCAGGTTGGCTCTGT
ATGGCTAAGCGTTTTCTCCTAAAATCCCTTGAAAACTGTGAGAAGACCAT
AAGAAGATCATATCTTTAATTCTATTTCACAAGTCACACAATATTCCAAT
CAAATACAGATGGTTGAGAAAAGTCATCCATCTTCCCTCCCCACCCTCCC
ACAGCCCCTCAACCACTGCCCTGAAACTTATATGCTGTTATCCGCAGCTC
CATCTGGAGCATCACAGCTACTGTCAACCCTGACGCTCTTTCTGAAAAAA
CACCGGATGGACATCAGAACTATTTCTTTAAGGATGTTACTGAGCCACAC
AGGAAAACTTGCCTTATGATTTTGAATGCACGGATCTGATTTGACTAAAC
ATGATAACTAGAGAATCACCCAATCTACTCCCATTTTCAACTCTAAATCA
TCAGAGTGTCTCAAATCCAAAGCACACACAGACCAGCCTGGCCAACACGG
TGAAACTCCACCCCTACTAAAAGTATAAAAATTATCCAGGTGTGGTGGCG
GGCGCCTGTAATCCAAGCTACTTGGGAGTCTGGAGGCAGGAGAATCCCTT
GAACCTGGGAGATGGAGGTTGCAGTGAGCAGAGATCACACCACCGCACTC
TAGCCTGGGCCACAAATCAACAACAACAACAACAACAAAAAACAAAGCGC
ACACAGAGACTGAGGTCCTCTTTGGCATTGAGAAGATGGCTATGCAAGTC
CCAACTAGCAAGTGCAAACTTCCCAGCTTCACTTCTGCCAGTGTCCCTTC
ACCCCTTCTCAACCCCACTGGGAGGCAGGAGGGTGCTTGACAATAACAGC
CTTGGCATCACTCTGCCAGGGTGTAATAGGAACTGTTACAATTCTGAGAT
TCTGTGTAAGCACTGGCCTTTCTGCCTAGAATGCCTTCTCCTCTCTTTTT
TAACTGCATGCTCCTATTTATCTTTCAAAGCCCGGAAAAAATAACACTGC
ACACGGGAAATGCTCCCTTCCTACTGCAGTCATTTAGATGACTCTATGCC
ATTCCATTCATTTCTCTTTCCTACCACAGAAGTGCTTTGAGATTTTGGAG
TCAGACTGCTTGAACTTGAATCCTGGCCCTCTCATCAGAGACTTGACTTA
TTTTAGGCAAGTTATATAACCAATTTTACCTCAGTTCCTTACCCATAAAA
TGGGTCTAATGAGAGTACCTACCACACAGAATTTTGATGAAAACTGAATG
AGATGAAGGCCTTTAAGGCAGTGGTCCCCAACCCTGGGGACACAGACAGG
TACCATTTTGTGGCCTGTTAGGAACTGGGCCACACAGCAGGAGGTGAGCA
GTGGGTGAGTGAGATCAGCGTTATTTACAGCTGCTCCCCATTGCTCACCT
TACTGCCTGAGCTCCACCTCCTGTCAGATCAGCAGTGGCATTAAATTCTC
ATAGCAGCACAAACCCTGTCATGAACTGCACATGCGAGGGATCTAGGTTG
TGCGCTCCTTATGAGAATCTAATGCCTAATGACCTGTCACCGTCTCCCAT
CACCCCTAGATGGGAGTGTCTAGTTGCAGGAAACAAGCTCAGGGCTTCCA
CTGATTCTACATTATGGTGAGTTGTATAATTATTTCATTATATAATACAA
TGTAATAATAATAGAAACACAGTGCACAACAAATGTAATGTGCTTGAATC
ATCCCCAAACCATCCCAGTCCACGGTCTTCCACATTTTGTCTTTTCACAA
AATTGTCTTCCACAAAACTGGTCCCTGGTGCCAAAAAGGCTTGGGACCAC
TGCTTTAAAGCCTTTGCATAGTGCTTAGAATTGAGGGGGAAAAAAAAAAC
AAAAACAATGTAGCTAGTTGCTACAATCACTATATTGGTGAGTTTCAAAA
GGAAAAGAATTCTGTCCCATTTATGCTTGAGCCTTGAGTTGCTAACCAAG
CCTGACACAAAATTACTGTTGAAGGGATGTGTGAGTCCTAATTGAAATGA
GGCCTCTTAAGGGAATTGTGGACCAAACCCCAAGCAGGCAGAAAGCCGTA
TCTTAATTATTGCAAGTATTTCAGGCAAGGTGTGGATGGCCATTTGAATT
CAAGCAGACTAGGACCTGGGATGAGAAAGAAGGTGTGTACGTGACTTGAT
CTTTGAACTTTAGCTCACCATCTGGAAGAAGGCTGAGTATTCTCTGCACT
CACATAGTAGCTAATGCCTACTCCCCAGCCACCCACAATTCTTTCTGTAG
GAAGGCTCGCTAGAATACTTTGTGATATTGGATATTAGTTCCATATTCTA
CTGTGTATCTTAGTTCAACCAAATTGTAATCATCTGATATTTATTTCTTT
TAATATAAATATAAGTATATTAAGTCTTAAAAAAAAAAAAAAAA

Real-Time RT-PCR

One μg of total RNA was used for reverse transcription in a 100 reaction of which 2 μl was used for PCR. All experiments were in duplicate or triplicate. PCR was performed for 45 cycles (denaturation: 95° C., 15 sec.; annealing/extension: 60° C., 1 min) with the following primers: forward primer: 5′-TCACTGACAACATCGACTTTGAAG (SEQ ID NO: 2); reverse primer: 5′-GGAGAAAACGCTTAGCCATACAG (SEQ ID NO: 3); probe (SEQ ID NO: 4): 6Fam-CTGGCCAGCCCTGCCACTTACCA-Tamra. Absolute quantification was done according to the manufacturers instructions (PE Biosystems, User Manual 2). The GAPDH gene was used as a reference gene. Accordingly, each sample was normalized according to its GAPDH content (level of expression) and also against a calibrator (SK-CO-10). The relative quantity was determined by the formula: 2^{(ΔCT}_{CCAT-1}^{-ΔCT}_{SK-CO-10}⁾. A standard curve corresponding to dilutions of CCAT-1 cDNA containing plasmid DNA was plotted and used to establish the quantity of CCAT-1 mRNA in SK− CO-10. This factor was multiplied with the relative quantity of each sample to obtain CCAT-1 quantity as fg per 100 ng cDNA. All experiments were performed using an ABI Prism 7000 system (Applied Biosystems, Foster City, Calif.).

### Example 2

### CCAT-1 Expression in Normal Tissues and Adenocarcinoma of the Colon and Rectum

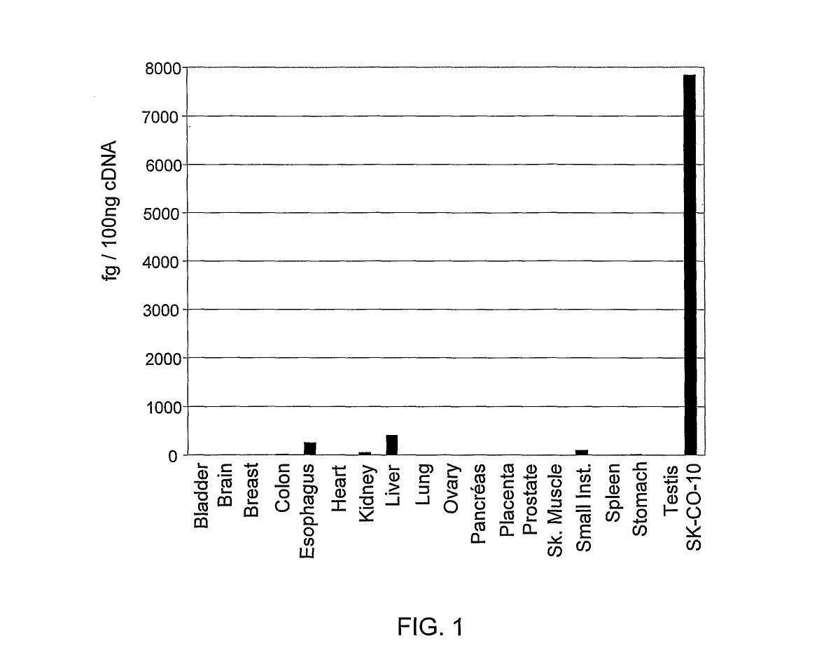

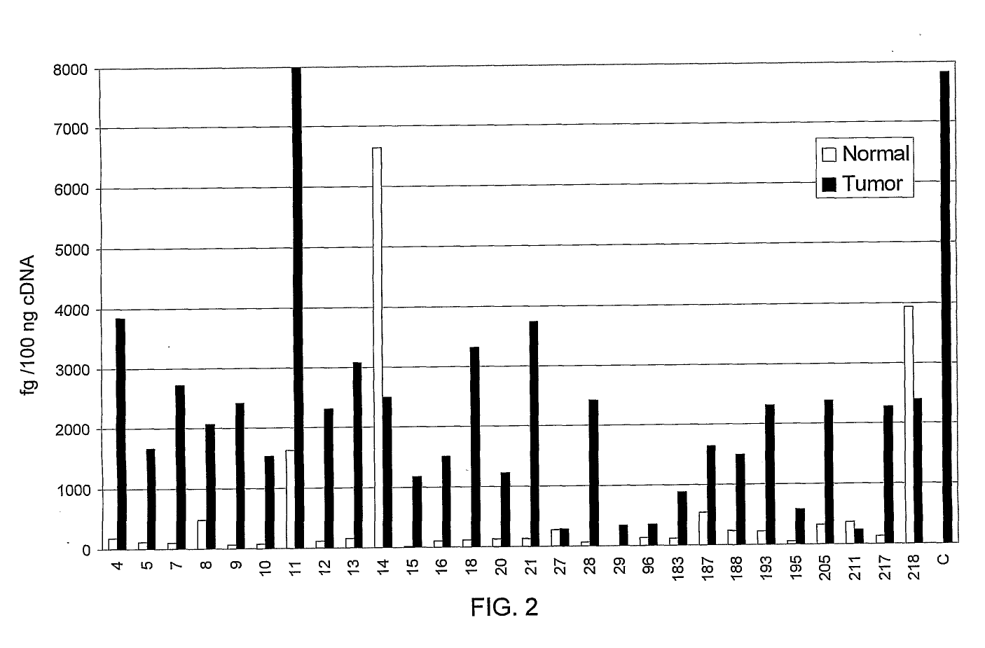

The expression of CCAT-1 was tested by quantitative real-time PCR in a panel of 18 normal tissues (FIG. 1). CCAT-1 expression ranged from 0.008 fg (skeletal muscle) to 402 fg in kidney. Compared to SK-CO-10, CCAT-1 levels in normal tissues were 20-fold (kidney), to more than 350-fold (colon) lower. In contrast, the average CCAT-1 mRNA level in tumor tissue was 2090 fg (p<0.0001, compared to normal colon), and ranged from 280 fg (27) to over 8000 fg (11), FIG. 2. In normal tissues adjacent to tumors the mean CCAT-1 mRNA level was 587 fg (p=0.04, compared to normal colon), and ranged from 0.1 fg (N29) to 6631 fg (N14), FIG. 2. In FIG. 2 paired samples of tumor and adjacent normal tissue were studied.

### Example 3

### Calibration curve of CCAT-1 in Peripheral Blood Mononuclear Cells (PBMCs)

All experiments in this part of the study were performed using an ABI Prism 7500 system (Applied Biosystems, Foster City, Calif.). In order to have a quantitative measurement of the CCAT-1 content in a given sample, a calibration curve was created. The colon cancer cell lines HT-29 and COLO-205 were obtained from ATCC (Manassas, Va.). Peripheral blood mononuclear cells, (PBMC) were obtained from peripheral blood of 10 healthy volunteers. PBMCs were separated by the Ficole gradient method and stored in −70° C. PBMCs and colon cancer cells were mixed in increasing concentrations of the colon cancer cells (1:1×10⁶, 1:5×10⁵, 1:1×10⁵, 1:5×10⁴, 1:1×10⁴, 1:5×10³, 1:1×10³, 1:500, 1:100, 1:50, 1:10, 1:1, and pure colon cancer cells).

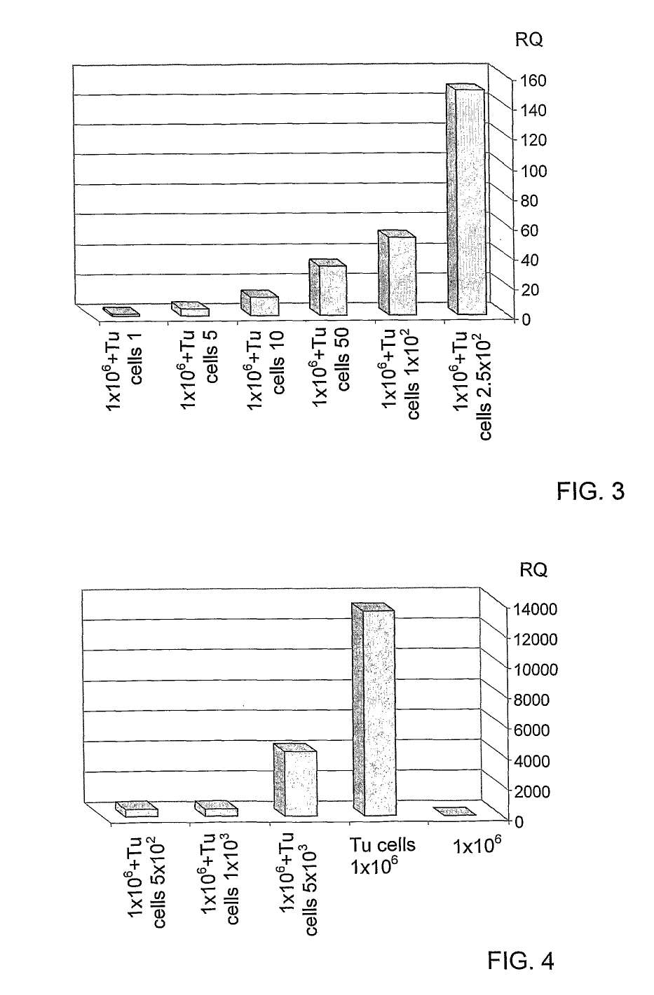

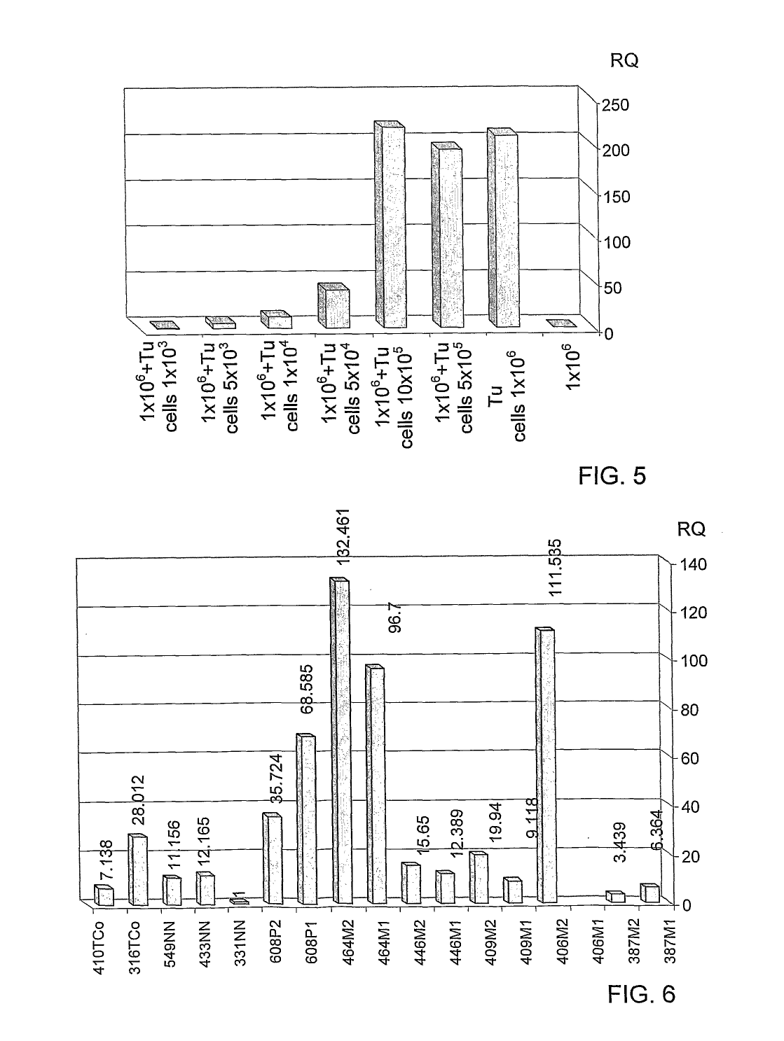

RNA was extracted and Real time PCR was performed for CCAT-1. In order to study the sensitivity of RT_PCR for CCAT-1 for the detection of small groups of cancer cells we created a calibration curve (FIGS. 3-5). Colon cancer cell line (HT29) cells in increasing concentrations were mixed with 10̂6 PBMCs. The lowest threshold for cancer cells detection was at a concentration of 5 cancer cells to 10̂6 PBMCs (FIG. 3).

### Example 4

### CCAT-1 Expression in Normal Colonic Mucosa, Pre-Malignant Mucosa, Adenomatous Polyps, Primary Adenocarcinoma of the Colon, and Distant Metastasis

Samples of normal colonic mucosa obtained from patients undergoing colonic resection for benign conditions, normal mucosa adjacent to the site of adenocarcinoma of the colon, adenomatous polyps, primary adenocarcinoma of the colon or rectum, and samples from liver or peritoneal metastasis, were snap frozen in liquid nitrogen. RNA was extracted from all tissue samples as previously described and real-time quantitative PCR for CCAT-1 was performed (FIG. 6).

### Example 5

### Detection of Occult Metastatic Disease in Blood and Lymph Nodes of Colon Cancer Patients

Patients

Patients over the age of 18 years with histologically confirmed primary adenocarcinoma of the colon were offered participation in the study. Patients with distant metastasis or patients who received prior radiation or chemotherapy were excluded from the study. The study protocol was approved by the Institutional Review Board (IRB, Helsinki Committee) Hadassah-Hebrew University Medical Center. Written informed consent was obtained from all study participants.

Sentinel Lymph Node Mapping and Harvest

Patients underwent standard surgical resection of adenocarcinoma of the colon including the normal wedge of mesentery containing the draining lymphatics. Immediately following removal of the surgical specimen (colon and mesentery), 2-5 ml of Isosulfan blue dye (Patent Blue V, France) was injected (ex vivo) subserosally in four quadrants around the tumor using a tuberculin syringe and needle. The en-bloc resection of tumor and lymph nodes was then evaluated on the back table and all blue nodes from the mesentery were dissected.

One half of the SLN(s) divided through the hilum along the longitudinal axis of the node was snap frozen in liquid nitrogen after ex vivo identification of the SLN(s). A small portion (˜25% of the entire tumor volume) of the primary tumor and a small sample (0.5 gr) of normal mucosa were snap frozen similarly. Separate instruments were used for the SLN(s) and primary tumor dissection ex vivo to minimize tumor cell contamination of the node(s).

Pathologic Processing and Cytokeratin Immunohistochemistry

The primary tumor specimen with attached mesentery and the remaining half of the SLN(s) were submitted to the Department of Pathology, Hadassah-Hebrew University Medical Center as separately labeled specimens. Once the diagnosis of colon adenocarcinoma was confirmed, formalin-fixed, paraffin-embedded blue nodes were subjected to serial step sectioning at four levels at an approximate thickness of 40 μm and were stained with H&E. In addition two unstained slides were prepared at the second and fourth level of the block for immunohistochemical staining, one for cytokeratin antibody staining, and the other to serve as the negative control. Immunohistochemistry was performed on formalin-fixed and paraffin-embedded sections of the SLN using the avidin-biotin-peroxidase complex method. A commercially obtained cytokeratin antibody cocktail was used in this study (Pan-keratin AE1/AE3, CAM 5.2, 35bH11, Ventana Medical Systems, Tucson, Ariz.). Endogenous peroxidase was suppressed by incubation with 1% hydrogen peroxide. Diaminobenzidine tetrahydrochloride (DAB, Biogenex, San Ramon, Calif.) was used as the chromogen. Formalin-fixed paraffin-embedded sections of tonsils were used as positive controls and a section from the SLN block was incubated with negative control buffer. A total of four H&E and two cytokeratin immunostained sections were examined for each block of tissue from the SLN. A cytokeratin immunostain was considered positive if strongly positive individual cells or cell clusters were identified that demonstrate anatomic and cytologic features of colon cancer cells.

### Example 6

### RT-PCR for Cytokeratin-20, CCAT-1 in Sentinel Lymph Nodes RNA Extraction

Extraction of total RNA was performed for all samples (tumors, lymph nodes, blood, and normal tissue) by the TriReagent method. Products were run in a gel for RNA quality evaluation prior to the synthesis of cDNA.

Frozen samples were powdered in dry ice, homogenized using a polytron homogenizer in trireagent (molecular research center, inc., Cincinnati, Ohio, usa) and processed according to the manufacturer's instructions.

Real-Time RT-PCR

One μg of total RNA was used for reverse transcription with random primers in a 20 μl reaction of which 2 μl was used for PCR. All experiments were in duplicate. PCR was performed for 40 cycles (denaturation: 95° C., 15 sec.; annealing/extension: 60° C., 1 min) with the primers and Taqman® probe specific for CK20 (Applied Biosystems, assay on demand). For CCAT-1 expression analysis, the primers used were as described above. Each sample was normalized according to its GAPDH content. The relative quantification was calculated against a calibrator for CK20 expression.

Since CCAT-1 negative samples were absolutely undetectable in lymph nodes and peripheral blood leukocytes, even by real time PCR, the relative quantity determination was irrelevant and the analyzed samples were simply evaluated as positive or negative for the expression of this transcript. All experiments in this part of the study were performed using an ABI Prism 7500 system (Applied Biosystems, Foster City, Calif.). RNA was extracted and Real time PCR was performed for CCAT-1.

CCAT 1 Expression in Sentinel Lymph Nodes of Colon Cancer Patients

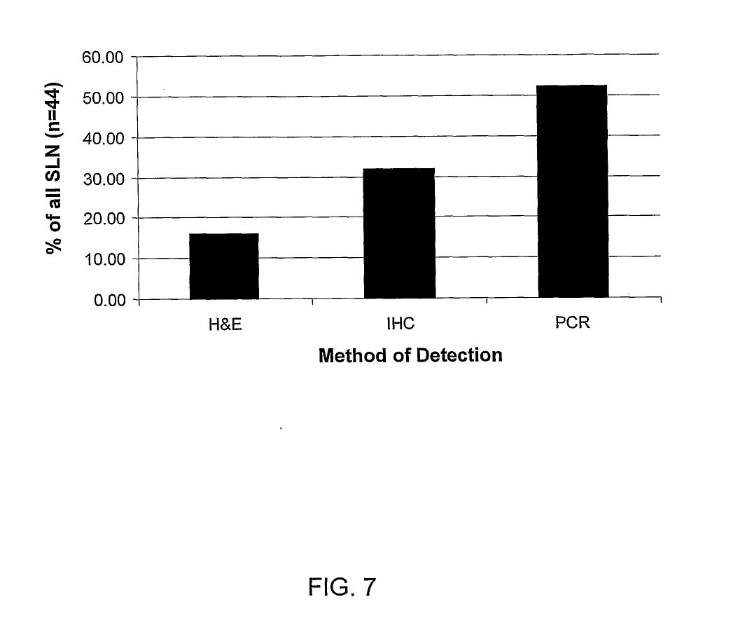

Real time PCR was conducted for all tumor and SLN tissues as well as 2 normal lymph nodes obtained from patients without malignancy and 3 PBMC samples obtained from healthy individuals. In all five negative controls there was no expression of CCAT-1 and only trace amounts of CK20. Therefore, “positive” PCR was set at RNA content of 50 times higher compared to the negative control value for the CK-20 values and since there was no amplification of CCAT-1 in the negative controls we used every SLN that had CCAT-1 content as positive. Sentinel lymph nodes harboring CRC metastasis were identified by H&E in 7/44 patients, by IHC for CK in 14/44 patients (p<0.0001 compared to H&E by chi-square test), and 23/44 by PCR (p=0.006 compared to H&E by chi-square test, FIG. 6, table 1). All H&E positive SLNs were also positive by IHC for CK and by PCR. However of the seven SLNs positive by IHC for CK only, four were positive also by PCR. Additional 12 SLNs negative by H&E and IHC were positive by PCR (FIG. 7).

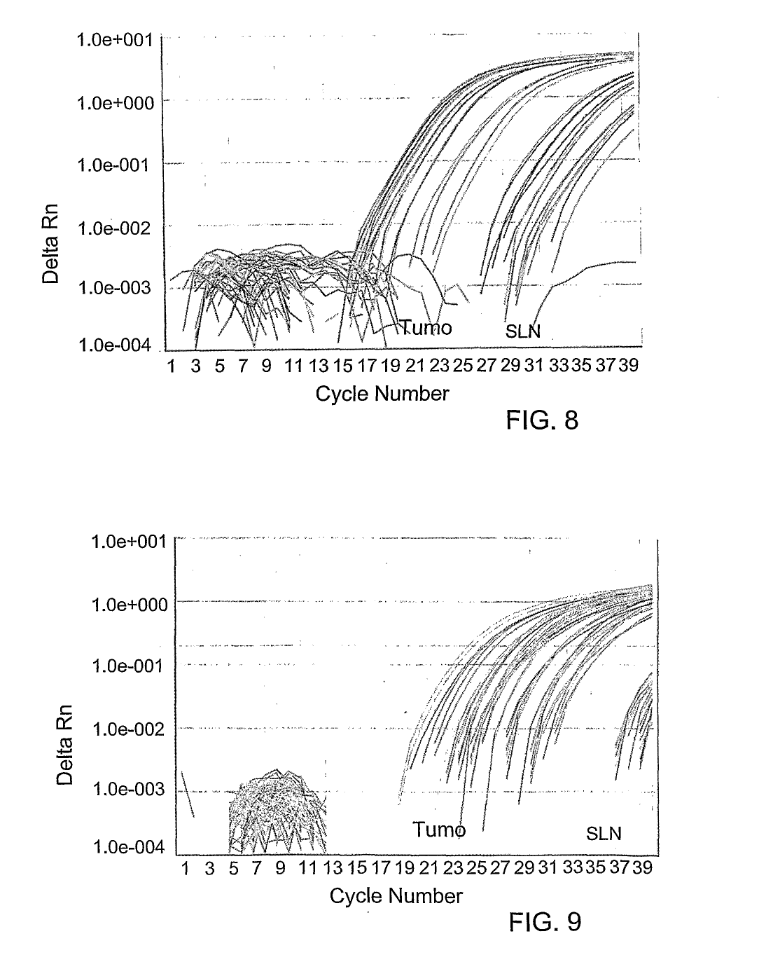

Amplification plots of qPCR for CK-20 and CCAT-1 are presented (FIGS. 8-9). Higher quantities of cDNA were amplified by CK-20 as compared to CCAT-1.

TABLE 1
Presence of CRC metastasis in SLNs by method of detection

| | | | | | CCAT-1 | CCAT-1 |
|---|---|---|---|---|---|---|
| Sample | H&E | IHC | CK tumor | CK SLN | tumor | SLN |
| 332 | neg | neg | Pos | pos | pos | neg |
| 456 | neg | neg | Pos | neg | pos | neg |
| 193 | neg | neg | Pos | neg | pos | neg |
| 195 | neg | neg | Pos | neg | pos | neg |
| 479 | neg | neg | Pos | neg | pos | neg |
| 480 | neg | neg | Pos | neg | pos | neg |
| 485 | pos | pos | Pos | pos | pos | pos |
| 400 | neg | neg | Pos | neg | pos | neg |
| 400 | neg | neg | Pos | neg | pos | neg |
| 491 | neg | neg | Pos | neg | pos | neg |
| 491 | neg | neg | Pos | neg | pos | neg |
| 491 | neg | neg | Pos | neg | pos | neg |
| 375 | neg | neg | Pos | pos | pos | neg |
| 360 | neg | neg | Pos | pos | pos | neg |
| 353 | neg | neg | Pos | pos | pos | neg |
| 307 | neg | neg | Pos | pos | pos | pos |
| 434 | pos | pos | Pos | neg | pos | pos |
| 434 | pos | pos | Pos | pos | pos | pos |
| 435 | neg | neg | Pos | pos | pos | pos |
| 435 | neg | neg | Pos | neg | neg | neg |
| 441 | neg | neg | Pos | pos | pos | neg |
| 447 | neg | pos | Pos | pos | pos | neg |
| 354 | neg | neg | Pos | pos | pos | neg |
| 381 | neg | neg | Pos | pos | pos | neg |
| 391 | neg | neg | Pos | neg | pos | neg |
| 361 | neg | neg | Pos | neg | pos | neg |
| 374 | neg | pos | Pos | pos | pos | neg |
| 395 | pos | pos | Pos | pos | pos | pos |
| 395 | neg | pos | Pos | neg | pos | neg |
| 358 | neg | pos | Pos | neg | pos | neg |
| 340 | neg | neg | Pos | neg | pos | neg |
| 340 | neg | neg | Pos | pos | pos | neg |
| 392 | neg | neg | Pos | neg | pos | neg |
| 404 | pos | pos | Pos | pos | pos | pos |
| 410 | neg | pos | Pos | neg | pos | neg |
| 313 | neg | neg | Pos | pos | pos | neg |
| 318 | neg | neg | Pos | pos | pos | neg |
| 373 | neg | pos | Pos | pos | pos | pos |
| 429 | neg | neg | Pos | neg | pos | neg |
| 397 | neg | pos | Pos | pos | pos | pos |
| 498 | neg | neg | Pos | neg | pos | neg |
| 498 | neg | neg | Pos | neg | pos | neg |
| 498 | pos | pos | Pos | pos | pos | pos |
| 501 | pos | pos | Pos | pos | pos | pos |
| LN | | neg | Neg | neg | neg | neg |
| LN | | neg | Neg | neg | neg | neg |
| Blood | | neg | Neg | neg | neg | neg |
| Blood | | neg | Neg | neg | neg | neg |
| Blood | | neg | Neg | neg | neg | neg |

### Example 7

### An RNA Based Stool Assay for the Early Detection of CRC Using CCAT-1

Since CCAT-1 negative samples were absolutely undetectable in lymph nodes and peripheral blood leukocytes, even by real time PCR, the relative quantity determination was irrelevant and the analyzed samples were simply evaluated as positive or negative for the expression of this transcript. All experiments in this part of the study were performed using an ABI Prism 7500 system (Applied Biosystems, Foster City, Calif.).

RNA was extracted and Real time PCR was performed for CCAT-1.

Stool Samples

Male or female patients over the age of 18 years presenting with a diagnosis of primary, non-metastatic (Clinical Stage I-III) colon carcinoma or patients scheduled to undergo full colonoscopy at the gastroenterology day-care were enrolled into this study.

Group 1-(N=15) Patients undergoing surgery for resection of histologically proven colon cancer;

Group 2-(N=15) Patients who underwent full colonoscopy without any tumor or polyp found at the colonoscopy.

Study Design

Stool samples were collected and immediately snap frozen in liquid nitrogen.

Total RNA from a histologically documented colon adenocarcinoma was used as the positive control for PCR. Negative PCR controls consist of PBMCs from healthy volunteers and reaction mixtures without template (internal controls).

Expression of CCAT-1 in Stool Samples of CRC Patients

Stool samples were collected from 15 patients with adenocarcinoma and 15 patients without adenocarcinoma.

RNA Extraction

Stool samples were obtained from all the study participants (n=30).

RNA may be extracted from fresh samples however such samples may not contain a sufficient quantity of RNA for analysis. Colonic washings from patients before colonoscopy or bowel preparation for colonic surgery may also be used for RNA extraction in sufficient quantity and quality. However, this method requires multiple concentrating procedures by centrifugation. A preferred method for RNA extraction was found to be by snap-freezing stool specimens in liquid nitrogen. This method yielded sufficient quantity of RNA for analysis. Various quantities of stool samples were analyzed with highest quantity and quality of RNA achieved from 150 mg of fresh frozen stool. Three different RNA extraction kits were compared with best results achieved by the Ambion 0 KIT. After finalizing the RNA extraction protocol RNA was successfully extracted from 12/15(80.0%) of study group samples and 9/15(60.0%) samples of the control group.

PCR Results

All three markers (A33, Co58, and CCAT-1) were initially studied in normal colon samples. Both A-33 and CO-58 were expressed in normal tissues and therefore we elected to focus on CCAT-1 as a single marker for our assay.

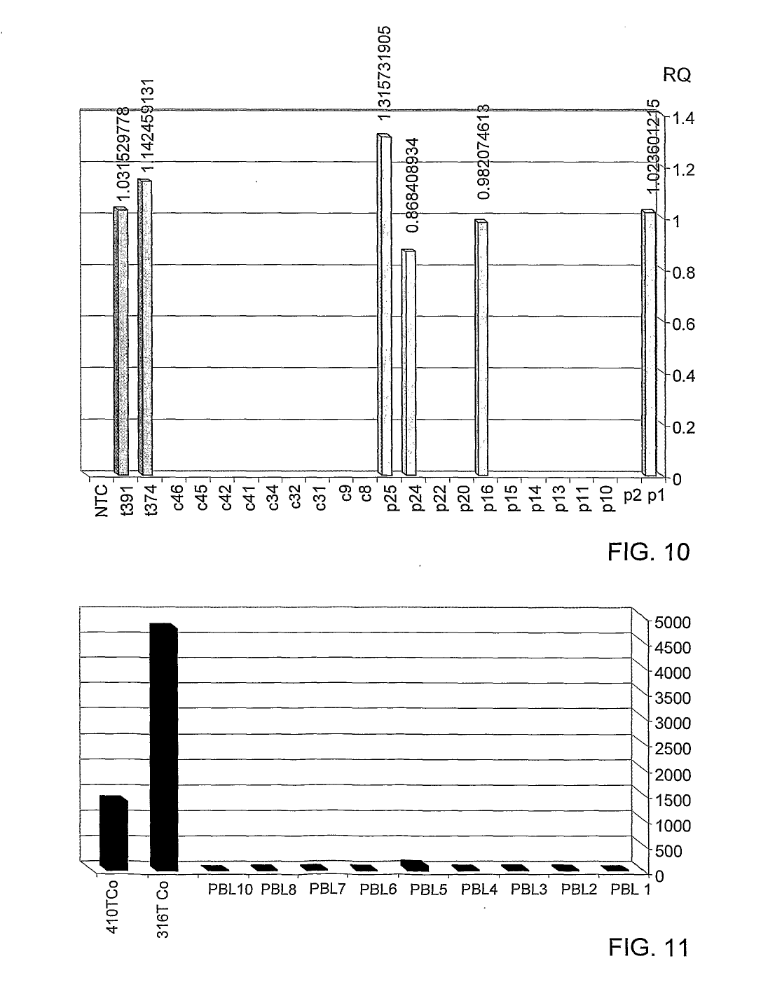

Real time (quantitative) PCR was performed on all samples having sufficient RNA (n=21). There was no evidence of CCAT-1 in stools of healthy individuals (n=9). There was significant expression of CCAT-1 in 4/12 (33.3%) of stool samples from CRC patients (p=000.1, Table 2 and FIG. 10).

TABLE 2
Clinical and molecular characteristics of CRC patients.

| | | | Anatomic | | | CCAT-1 |
|---|---|---|---|---|---|---|
| Study # | Age | Gender | location | Stage | Grade | expression |
| P1 | 77 | M | Lt + Rt | 3 | 2 | + |
| P2 | 70 | F | Lt | 2 | 2-3 | − |
| P10 | 52 | M | Lt | 1 | 1 | − |
| P11 | 65 | F | Rectum | 3 | 1-2 | − |
| P13 | 81 | F | Lt | 3 | 1-2 | − |
| P14 | 61 | F | Lt | 3 | 2 | − |
| P15 | 77 | M | Lt | 3 | 1-2 | − |
| P16 | 85 | F | Rectum | 3 | 2 | + |
| P20 | 79 | M | Rectum | 3 | 2-3 | − |
| P22 | 57 | F | Lt | 2 | 1 | − |
| P24 | 74 | M | Rectum | 2 | 2 | + |
| P25 | 66 | M | Lt | 3 | 2 | + |

### Example 8

### The Presence of CCAT-1 in Peripheral Blood of Colon Cancer Patients

Blood Samples

15 ml of blood were obtained from the patients participating in the study (n=20). The blood samples were separated by the Ficole gradient method and stored in −70° C. RNA was extracted and Real time PCR was performed for CCAT-1. Results were compared to the calibration curve.

Expression of CCAT-1 in Peripheral Blood of Colon Cancer Patients

In order to study the sensitivity of RT-PCR for CCAT-1 for the detection of small numbers of cancer cells we created a calibration curve.

Colon cancer cell line (HT29) cells in increasing concentrations were mixed with 10⁶ PBMCs. The lowest threshold for cancer cells detection was at a concentration of 50 cancer cells to 10⁶ PBMCs (FIGS. 3-5).

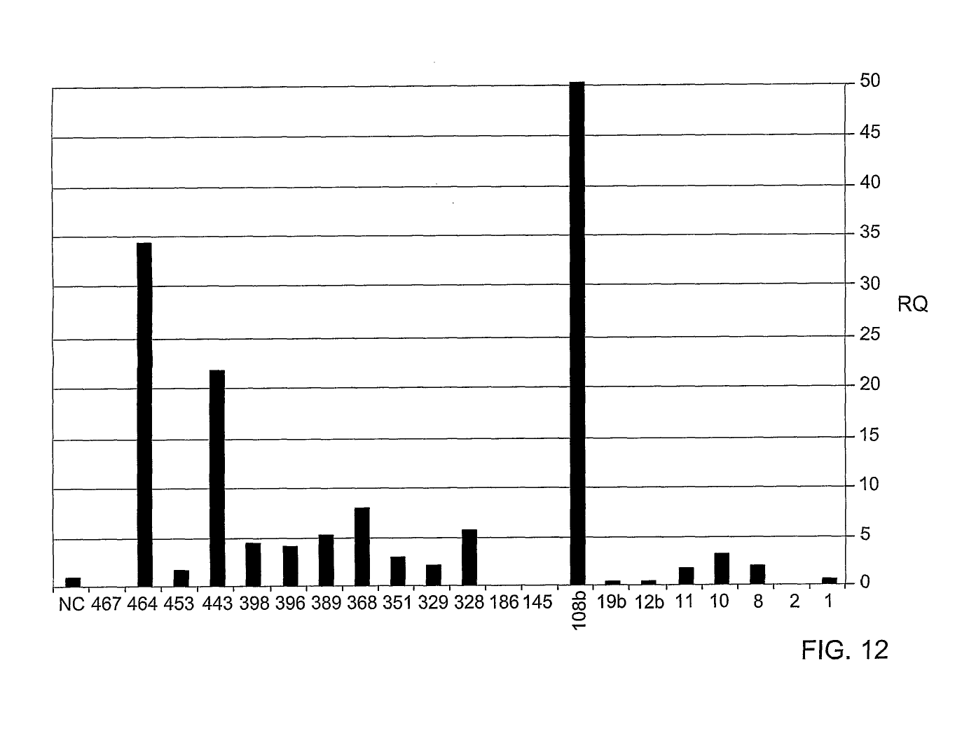

As can be seen in FIG. 11, no CCAT-1 expression can be detected in peripheral blood samples of healthy individuals. In contrast, various degrees of CCAT-1 expression can be easily detected in peripheral blood samples of colon cancer patients (FIG. 12).

### Example 9

### CCAT-1 Expression in Additional Colorectal Cancer Cell Lines

The expression of CCAT-1 was examined in a wide range of colorectal cancer (CRC) cell lines. Eighteen CRC cell lines were selected for the experiment. In the present context, HT-29 was set as a reference for CCAT-1 expression. RNA was extracted form all cell lines and cDNA was created using established methods. Real-time quantitative PCR, i.e. qPCR, was used to measure the relative CCAT-1 expression in this group of CRC cell lines.

TABLE 3
Real-time (quantitative) PCR results of CCAT-1 expression in 18
CRC cell lines as compared to HT-29.

| CRC Cell line | Avg Ct |
|---|---|
| HT 29 | 25.2 |
| CaCO2 | 20.49 |
| HCT15 | 21.102 |
| LS 174T | 20.974 |
| SK-CO-1 | 19.739 |
| SW 403 | 19.725 |
| SW 1222 | 19.584 |
| SW 620 | 21.282 |
| SW 802 | 19.901 |
| SW 837 | 18.677 |
| CO 61 | 19.613 |
| SK-CO-11 | 18.461 |
| SK CO 10 | 20.025 |
| SW1083 | 21.026 |
| HT29 | 26.229 |
| LOVO | 21.04 |
| WS1116 | 21.366 |
| SW48 | 20.244 |
| SK-CO-17 | 20.34 |

Average Ct=PCR cycle of amplification (the lower the cycle the higher the specific cDNA content in the sample, higher CCAT-1 expression).

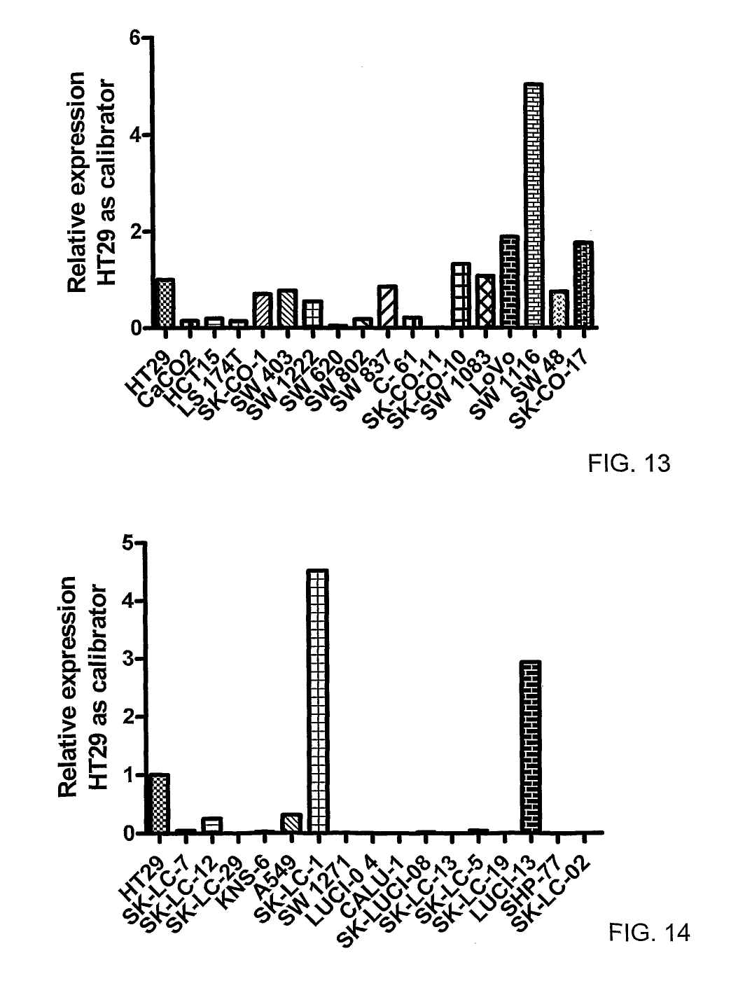

FIG. 13 shows relative CCAT-1 expression of 18 colorectal cancer cell lines compared to HT-29. The relative CCAT-1 expression in any given sample was compared to CCAT-1 expression in HT-29 (=1). The relative expression value was calculated from the average Ct difference between a given sample, a house keeping gene (GPADH) in the sample, and the HT-29 sample.

### Example 10

### CCAT-1 Expression in Cell Lines Representing Cancers Other than Colorectal Cancer

In order to exemplify CCAT-1 expression in other types of human cancer, multiple cell lines representing several common human cancers were screened by qPCR for CCAT-1 expression.

Non-Small Cell Lung Cancer

Non-small cell lung cancer (NSCLC) is the most common form of human lung cancer. It is still the main cause of cancer-related death worldwide.

Expression of CCAT-1 was studies in human tumor tissues from NSCLC patients vs. corresponding normal lung tissues. Sixteen cell lines representing NSCLC were studied as well. CCAT-1 expression was determined by quantitative PCR analysis.

TABLE 4
Real-time (quantitative) PCR results of CCAT-1 expression in 16 lung
cancer cell lines

| Sample | Avg Ct | 2-ddCt | |
|---|---|---|---|
| HT 29 | 25.5 | 1 | colon calibrator |
| 612T | 25.356 | 0.3175 | NSCLC tumor tissue |
| 612N | 0 | | corresponding lung tissue |
| 615T | 30.377 | 0.0117 | NSCLC tumor tissue |
| 615N | 36.17 | 8E−05 | corresponding lung tissue |
| 618T | 37.549 | 6E−05 | NSCLC tumor tissue |
| 618N | 37.407 | 0.0001 | corresponding lung tissue |
| 629T | 29.953 | 0.0081 | NSCLC tumor tissue |
| 629N | 32.276 | 0.003 | corresponding lung tissue |
| sk-lc-7 | 25.769 | 0.0485 | NSCLC |
| sk-lc-29 | 0 | | NSCLC |
| KNS-6 | 25.355 | 0.0177 | NSCLC |
| A549 | 25.477 | 0.308 | NSCLC |
| sk-lc-1 | 26.867 | 4.5159 | NSCLC |
| sk-lc-12 | 25.091 | 0.2512 | NSCLC |
| SW1271 | 0 | | NSCLC |
| Luci4 | 31.842 | 0.003 | NSCLC |
| calu1 | 0 | | NSCLC |
| sk-luci-8 | 27.729 | 0.0273 | NSCLC |
| sk-lc-13 | 0 | | NSCLC |
| sk-lc-5 | 28.378 | 0.0485 | NSCLC |
| sk-lc-19 | 0 | | NSCLC |
| Luci-13 | 20.259 | 2.9302 | NSCLC |
| SHP-77 | 31.673 | 0.0003 | NSCLC |
| sk-lc-2 | 36.392 | 0.0002 | NSCLC |

Average Ct ═PCR cycle of amplification (the lower the cycle the higher the specific cDNA content in the sample, higher CCAT-1 expression). 2-ddCt.=The Log difference calculated between the house keeping gene (GPADH) Ct, the HT-29 Ct and the sample of interest Ct. Over-expression of CCAT-1 was measured in 2/16 cell lines (12.5%), the 2 cell lines are sk-lc-1 and Luci-13 (see Table 4).

CCAT-1 expression in NSCLC cell lines: Relative expression was calculated by the difference in Ct between a housekeeping gene, HT-29 (=1) and the sample of interest.

FIG. 14 shows relative CCAT-1 expression of 16 NSCLC cancer cell lines compared to HT-29. The relative CCAT-1 expression in any given sample was compared to CCAT-1 expression in HT-29 (=1). The relative expression value was calculated from the average Ct difference between a given sample, a house keeping gene (GPADH) and the HT-29 sample.

### Example 11

### CCAT-1 Expression in Breast Cancer Cell Lines

Breast cancer is a leading cause of cancer-related mortality in women. Despite major advances in diagnosis and therapy, overall outcome of patients with advanced stages is poor. Novel targets for diagnosis, staging, and treatment are important to improve outcome in both diseases. Therefore, 13 cell lines representing breast cancer were screened for the expression of CCAT-1 using qPCR.

TABLE 5
Real-time (quantitative) PCR results of CCAT-1 expression in
breast cancer cell lines

| Cell Line | Avg Ct | 2-ddCt | |
|---|---|---|---|
| HT 29 1-3-08 | 25.5 | 1 | colon calibrator |
| MDA MB 468 | 35.2075 | 7E−05 | breast |
| sk-br-5 | 0 | 0 | breast |
| HTB | 0 | 0 | breast |
| BTOO | 0 | 0 | breast |
| CAMA | 33.871 | 9E−05 | breast |
| MCF7 | 37.573 | 1E−05 | breast |
| SK-BR-3 | 35.426 | 2E−04 | breast |
| HCC1954 | 23.373 | 0.054 | breast |
| MDA MB 231 | 31.316 | 0.002 | breast |
| MDA MB 175 | 0 | 0 | breast |
| BT20 | 35.033 | 3E−05 | breast |
| BT474 | 36.782 | 9E−06 | breast |
| MDA MB 453 | 0 | 0 | breast |

Average Ct = PCR cycle of amplification (the lower the cycle the higher the specific cDNA content in the sample, higher CCAT-1 expression).
2-ddCt. = The Log difference calculated between the house keeping gene(GPADH) Ct, the HT-29 Ct and the sample of interest Ct.

There was no significant CCAT-1 expression in the 13 breast cancer cell lines studied. In addition, CCAT-1 expression was studied also in 6 melanoma cell lines. None of the melanoma cell lines showed significant CCAT-1 expression.

### Example 12

### In Situ Hybridization Analysis

In order to validate the expression of CCAT-1 in colon cancer tissues and study the morphological pattern of expression, in-situ hybridization was performed.

Slides from paraffin blocks containing tumor tissue (n=2) and adjacent normal mucosa (n=2) were used for staining.

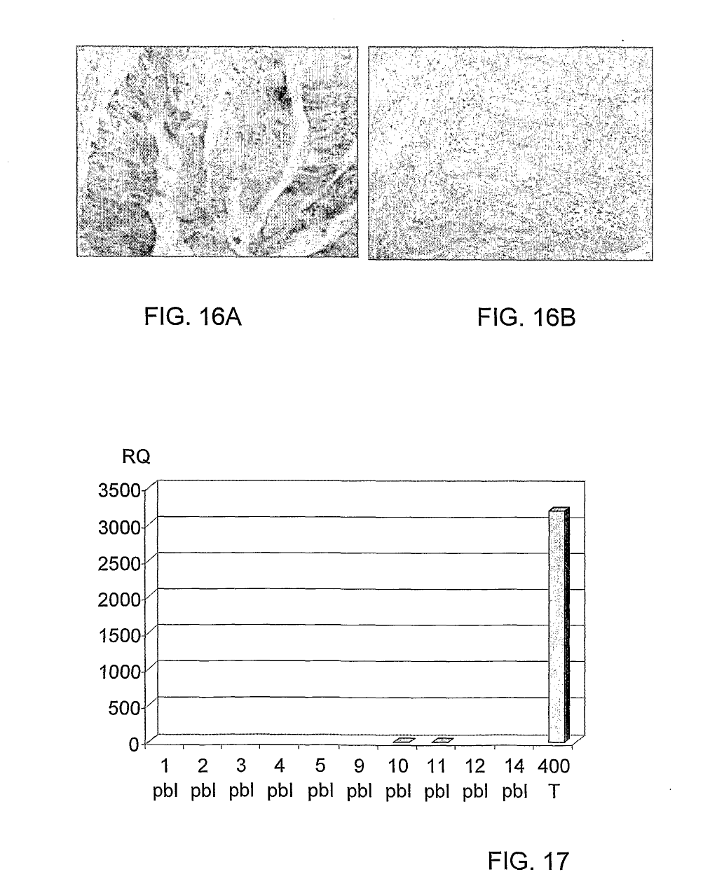

A probe for CCAT-1 (shown in FIG. 16A; SEQ ID NO. 5) was prepared for use in the analysis of the morphological pattern of CCAT-1 expression having the following sequence:

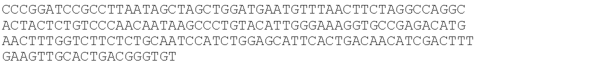

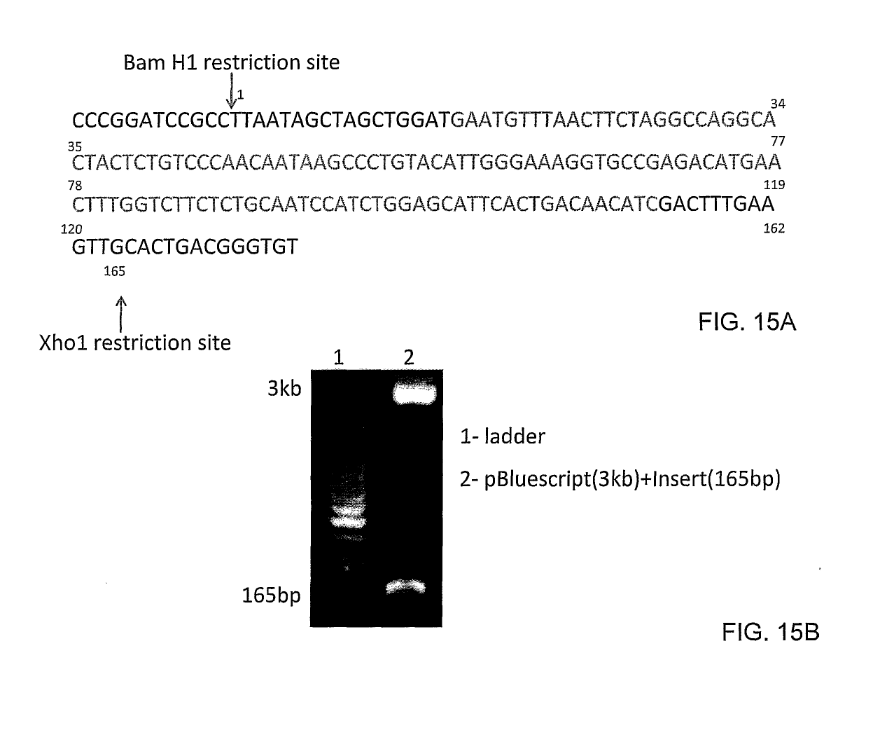

Since the gene is not translated a probe was cloned in two different plasmids in order to create sense and anti-sense probes. Antisense RNA probe was synthesized with the insert into pBluescript-KS (pBSKS, bacterial vector 3 Kbp long) by T7 RNA polymerase, and the sense RNA probe was synthesized with the insert into pBluescript-SK (pBSKS, bacterial vector 3 Kbp long) by T7 RNA polymerase. The size of the insert was 165 bp (FIG. 15). The two plasmids were:

1. pBluescript-SK (+) [pBSSK+], bacterial vector 3 Kbp long.

2. pBluescript-KS (−) [pBSKS−], bacterial vector 3 Kbp long.

These vectors are commercially available, for example by Stratagene, Calif. It should be understood that other vectors and alike can be utilizes in this respect.

FIG. 16B exemplifies In situ hybridization based screening in which staining of CCAT-1 in adenocarcinoma of the colon (a) and in adjacent normal mucosa (b) is shown. Stronger CCAT-1 staining is seen in the tumor tissue (a) as compared to a lower intensity if the CCAT-1 staining in adjacent normal mucosa. This correlates with the real-time PCR results showing low CCAT-1 expression in normal mucosa adjacent to the tumor.

### Example 13

### CCAT-1 Expression in Peripheral Blood of Healthy Volunteers

The expression of CCAT-1 in blood samples of healthy volunteers (n=10) was also studied.

FIG. 17 is a graph showing expression of CCAT-1 in peripheral blood samples of healthy volunteers. Relative quantity of CCAT-1 cDNA (expression) was undetected in all blood samples. As expected, there was strong CCAT-1 amplification at the T-400 sample containing tumor tissue as a positive control.

### REFERENCES

1. Cancer statistics, 2000. Greenlee RT—CA Cancer J Clin—2000 January-February; 50(1): 7-33.
2. Midgley R, Kerr D. Colorectal cancer. Lancet 1999; 353:391-399.
3. Cohen Am, Kelsen D, Slatz L, et al. Adjuvant therapy for colorectal cancer. Curr Prob Cancer 1998; 22:5-65.

1. A method of diagnosing cancer or precancerous lesions, comprising measuring the level of expression of SEQ ID NO: 1 (CCAT-1) or a fragment thereof in a biological sample; wherein expression of SEQ ID NO: 1 (CCAT-1) or a fragment thereof in the biological sample, is indicative of cancer or a precancerous lesion.

2. The method of diagnosing cancer or precancerous lesions according to claim 1, wherein said method further comprises comparing said expression level measured in the biological sample with a standard, wherein a higher level of expression of SEQ ID NO: 1 (CCAT-1) or a fragment thereof in the biological sample, is indicative of cancer or a precancerous lesion.

3. The method of diagnosing cancer or a precancerous lesion in accordance with claim 2, comprising:
(a) isolating nucleic acids from a biological sample obtained from a subject;
(b) hybridizing a probe capable of recognizing CCAT-1 with said nucleic acids, under conditions allowing the formation of hybridization complexes; and
(c) comparing hybridization complex formation with a standard; wherein a higher level of hybridization complexes in the biological sample is indicative of cancer or a precancerous lesion.

4. The method of diagnosing cancer or a precancerous lesion in accordance with claim 2, comprising:
(a) isolating nucleic acids from a biological sample obtained from a subject;
(b) amplifying CCAT-1 or any fragment thereof in said isolated nucleic acids;
(c) visualizing the CCAT-1 amplified product; and
(d) comparing the amount of CCAT-1 amplified product with a standard;
wherein the presence of a higher level of a CCAT-1 amplified product is indicative of cancer or a precancerous lesion.

5. The method according to claim 4 wherein said amplification is performed by polymerase chain reaction (PCR) using CCAT-1 specific probes.

6. The method according to claim 5, wherein said PCR is a real-time quantitative PCR.

7. The method according to claim 2 wherein said standard is determined by measuring the level of expression of CCAT-1 in a subject not afflicted with cancer.

8. The method according to claim 2 wherein said standard is determined by measuring the level of expression of CCAT-1 in a non-cancerous tissue of said same subject.

9. The method of claim 1, wherein the cancer is selected from the group consisting of: colon cancer, rectal cancer, lung cancer, and metastases of said cancers.

10. The method of claim 1, wherein the precancerous lesion is an adenomatous polyp.

11. The method of claim 1, wherein said biological sample is selected from the group consisting of tissue, blood, urine, stool, and bone marrow samples.

12. (canceled)

13. (canceled)

14. An isolated oligonucleotide comprising at least 8 contiguous nucleotides of SEQ ID NO: 1 (CCAT-1) or a complement thereof, useful as a probe or a primer.

15. (canceled)

16. The oligonucleotide probe of claim 14, wherein the probe comprises SEQ ID NO: 5.

17. (canceled)

18. (canceled)

19. A method for detecting the expression of CCAT-1 in a biological sample comprising:
(a) isolating nucleic acids from said biological sample;
(b) hybridizing the oligonucleotide probe of claim 16 to said nucleic acids under conditions allowing the formation of hybridization complexes; and
(c) comparing hybridization complex formation with a standard, wherein a higher level of hybridization complexes in the biological sample indicates expression of CCAT-1 in the sample.

20. (canceled)

21. An isolated nucleic acid comprising CCAT-1 or a fragment thereof having at least 85% homology with SEQ ID NO: 1.

22. The isolated nucleic acid according to claim 21 comprising the nucleic acid sequence of SEQ ID NO: 1 or a fragment thereof.

23. (canceled)

24. A vector comprising the isolated nucleic acid of claim 21.

25. A host cell comprising the vector of claim 24.

26. A composition according to claim 23 wherein the composition is attached to a substrate.

27. (canceled)

28. (canceled)

29. A method of imaging cancer or precancerous lesions, comprising:
(a) administering to a subject a probe in accordance with claim 14;
wherein said probe is conjugated to an indicator molecule; and
(b) detecting the indicator molecule conjugated to said probe by an imaging device.

30. (canceled)
